(19) United States
(12) Patent Application Publication
(10) Pub. No.: US 2022/0290990 A1
(43) Pub. Date: Sep. 15, 2022

(54) MEASURING METHOD, MEASURING APPARATUS AND PROGRAM

(71) Applicant: NIPPON TELEGRAPH AND TELEPHONE CORPORATION, Tokyo (JP)

(72) Inventors: Hideyuki TSUBOI, Musashino-shi, Tokyo (JP); Hideki TOSHINAGA, Musashino-shi, Tokyo (JP); Kazuto GOTO, Musashino-shi, Tokyo (JP); Satoshi KUROSAKI, Musashino-shi, Tokyo (JP); Mizuki SUGA, Musashino-shi, Tokyo (JP); Kota ITO, Musashino-shi, Tokyo (JP); Naoki KITA, Musashino-shi, Tokyo (JP)

(73) Assignee: NIPPON TELEGRAPH AND TELEPHONE CORPORATION, Tokyo (JP)

(21) Appl. No.: 17/625,506

(22) PCT Filed: Jul. 10, 2019
(86) PCT No.: PCT/JP2019/027274

§ 371 Date: Jan. 7, 2022

(51) Int. Cl. G01C 15/00; G01S 17/89; G01B 11/24; G01N 33/00

(52) U.S. Cl. CPC G01C 15/002; G01S 17/89; G01B 11/24; G01N 33/0098

(57) ABSTRACT

A measurement method includes: a data acquisition step of acquiring position data output from a measurement device configured to move round-trip on a movement route and measure a position of an object in a periphery of the movement route, the measurement device using a plane having a depression angle or an elevation angle with respect to a plane that is orthogonal to a forward direction as a measurement target; and a point group data generation step of generating point group data for an object in the periphery of the movement path using the position data obtained during movement of the measurement device on an outbound trip and position data obtained during movement of the measurement device on a return trip.

### TECHNICAL FIELD

The present invention relates to a measurement method, a measurement apparatus, and a program.

### BACKGROUND ART

There is an MMS (Mobile Mapping System) in which various types of measurement devices are combined and mounted in a vehicle, measurement is performed while travelling on a road, and three-dimensional data (e.g., point group data) of objects in the surrounding area is generated. A measurement device such as a laser radar (LIDAR: Light Detection and Ranging) for performing measurement of the direction in which a target object is present and the distance from the vehicle to the target object is mounted in the MMS (e.g., see NPL 1). Conventionally, effective maintenance and inspection of the deterioration state of outdoor infrastructure such as electrical poles for communication have been performed by analyzing collected three-dimensional data (e.g., see PTL 1 and NPL 2).

### CITATION LIST

### Patent Literature

[PTL 1] Japanese Patent Application Publication No. 6531051
[PTL 2] Japanese Patent Application Publication No. 5870011
[PTL 3] Japanese Patent Application Publication No. 6381137

### Non-Patent Literature

[NPL 1] M. Waki, T. Goto, K. Katayama, “Review Paper: 3D Facility Management Technology Using Mobile Mapping System”, Communication Society Magazine, No. 45 Summer Issue, pp. 39-45, IEICE, 2018
[NPL 2] “Mitsubishi Mobile Mapping System High-precision GPS Mobile Measuring Equipment”, product pamphlet, Mitsubishi Electric Corporation, March 2016

### SUMMARY OF THE INVENTION

### Technical Problem

Incidentally, examples of measurement targets with complex shapes include trees and the like. In a tree, leaves growing at positions on the forward direction side and the opposite direction side of the MMS in particular often grow in a direction with their side surfaces facing the MMS. In general, due to the fact that it is difficult to perform measurement at an interval that is narrow enough to be equal to the thickness of a leaf on a tree, it is difficult to measure a leaf growing facing to the side without omission. For this reason, on a tree, regions in which measurement through MMS is difficult (hereinafter referred to as “measurement-undetected regions”) occur in a range on the forward direction side and the opposite direction side of the MMS in particular in some cases. In this case, due to the measurement-undetected regions being generated, there is a problem in that there is a risk that the measured width of the tree will be recognized as a width that is narrower than the actual width by an amount corresponding to the measurement-undetected regions.

The present invention was made in view of the above-described circumstance, and aims to provide a measurement method, a measurement apparatus, and a program according to which it is possible to reduce the measurement-undetected regions.

### Means for Solving the Problem

An aspect of the present invention is a measurement method including: a data acquisition step of acquiring position data output from a measurement device configured to move round-trip on a movement route and measure a position of an object in a periphery of the movement route, the measurement device using a plane having a depression angle or an elevation angle with respect to a plane that is orthogonal to a forward direction as a measurement target; and a point group data generation step of generating point group data for an object in the periphery of the movement path using the position data obtained during movement of the measurement device on an outbound trip and the position data obtained during movement of the measurement device on a return trip.

Also, an aspect of the present invention is a measurement apparatus including: a data acquisition unit configured to acquire position data output from a measurement device configured to move round-trip on a movement route and measure a position of an object in a periphery of the movement route, the measurement device using a plane having a depression angle or an elevation angle with respect to a plane that is orthogonal to a forward direction as a measurement target; and a point group data generation unit configured to generate point group data for an object in the periphery of the movement path using the position data obtained during movement of the measurement device on an outbound trip and the position data obtained during movement of the measurement device on a return trip.

Also, an aspect of the present invention is a program for causing a computer to execute the above-described measurement method.

### Effects of the Invention

With the present invention, it is possible to reduce measurement-undetected regions.

### BRIEF DESCRIPTION OF DRAWINGS

FIG. 1 is a schematic diagram showing an acquisition position of position data acquired by a conventional MMS.

FIG. 2 is a schematic diagram showing the acquisition position of position data acquired by a conventional MMS.

FIG. 3 is a schematic diagram showing the acquisition position of position data acquired by a conventional MMS.

FIG. 4 is a schematic diagram showing an example of measurement performed by a conventional MMS.

FIG. 5 is a schematic diagram showing an example of measurement performed by a conventional MMS.

FIG. 6 is a schematic diagram showing an example of measurement performed by a conventional MMS.

FIG. 7 is a schematic diagram showing a relationship between a distance to a measurement target and an interval of position data acquisition positions.

FIG. 8 is a schematic diagram showing the size of a space that can be measured by a conventional MMS.

FIG. 9 is a schematic diagram showing measurement-undetected regions in the case where the measurement target is a tree.

FIG. 10 is a schematic diagram showing measurement-undetected regions in the case where the measurement target is a tree.

FIG. 11 is a schematic diagram showing a relationship between an orientation of a leaf Lf and an emission direction of laser light emitted by a laser radar.

FIG. 12 is a schematic diagram showing a relationship between an orientation of a leaf Lf and an emission direction of laser light emitted by a laser radar.

FIG. 13 is a schematic diagram showing a relationship between an orientation of a leaf Lf and an emission direction of laser light emitted by a laser radar.

FIG. 14 is a schematic diagram showing a relationship between an orientation of a leaf Lf and an emission direction of laser light emitted by a laser radar.

FIG. 15 is a schematic diagram showing a relationship between an orientation of a leaf Lf and an emission direction of laser light emitted by a laser radar.

FIG. 16 is a schematic diagram showing a relationship between an orientation of a leaf Lf and an emission direction of laser light emitted by a laser radar.

FIG. 17 is a schematic diagram showing acquisition positions of position data acquired by an MMS according to a first embodiment of the present invention.

FIG. 18 is a schematic diagram showing acquisition positions of position data acquired by the MMS according to the first embodiment of the present invention.

FIG. 19 is a schematic diagram showing acquisition positions of position data acquired by the MMS according to the first embodiment of the present invention.

FIG. 20 is a schematic diagram showing an example of measurement performed by the MMS according to the first embodiment of the present invention.

FIG. 21 is a schematic diagram showing an example of measurement performed by the MMS according to the first embodiment of the present invention.

FIG. 22 is a schematic diagram showing an example of measurement performed by the MMS according to the first embodiment of the present invention.

FIG. 23 is a schematic diagram for illustrating a measurement-undetected region in the case where a measurement target is a tree.

FIG. 24 is a schematic diagram for illustrating measurement-undetected regions in the case where a measurement target is a tree.

FIG. 25 is a schematic diagram showing a relationship between an orientation of a leaf Lf and an emission direction of laser light emitted by a laser radar.

FIG. 26 is a schematic diagram showing a relationship between an orientation of a leaf Lf and an emission direction of laser light emitted by a laser radar.

FIG. 27 is a schematic diagram showing a relationship between an orientation of a leaf Lf and an emission direction of laser light emitted by a laser radar.

FIG. 28 is a flowchart showing operations of the MMS according to the first embodiment of the present invention.

FIG. 29 is a schematic diagram for illustrating coordinate alignment processing performed by the MMS according to the first embodiment of the present invention.

FIG. 30 is a schematic diagram for illustrating coordinate alignment processing performed by the MMS according to the first embodiment of the present invention.

FIG. 31 is a schematic diagram for illustrating point group data overlaying processing.

FIG. 32 is a schematic diagram for illustrating point group data overlaying processing.

FIG. 33 is a schematic diagram for illustrating point group data overlaying processing.

FIG. 34 is a schematic diagram for illustrating point group data overlaying processing.

FIG. 35 is a block diagram showing a functional configuration of the MMS according to the first embodiment of the present invention.

FIG. 36 is a schematic diagram showing acquisition positions of position data acquired by an MMS according to a second embodiment of the present invention.

FIG. 37 is a schematic diagram showing acquisition positions of position data acquired by the MMS according to the second embodiment of the present invention.

FIG. 38 is a schematic diagram showing acquisition positions of position data acquired by the MMS according to the second embodiment of the present invention.

FIG. 39 is a flowchart showing operations of the MMS according to the second embodiment of the present invention.

FIG. 40 is a schematic diagram for illustrating coordinate alignment processing performed by an MMS according to a third embodiment of the present invention.

FIG. 41 is a schematic diagram for illustrating coordinate alignment processing performed by the MMS according to the third embodiment of the present invention.

### DESCRIPTION OF EMBODIMENTS

Hereinafter, first, a measurement method performed using a conventional technique will be described in order to facilitate comprehension of the description of the embodiment of the present invention.

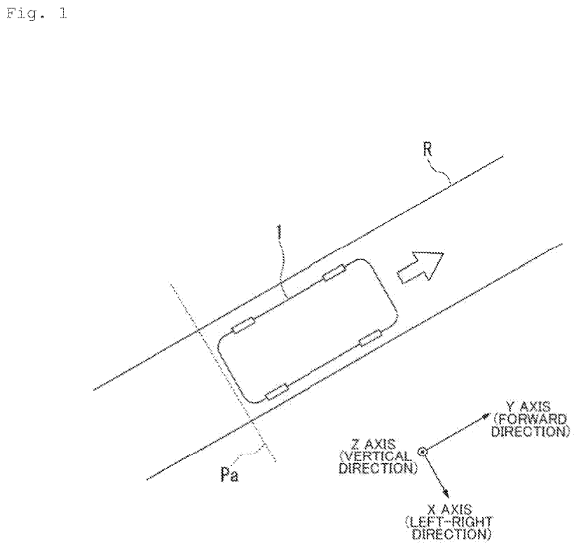

FIGS. 1 to 3 are schematic diagrams showing acquisition positions of position data acquired by a conventional MMS. FIGS. 1 to 3 respectively show a plan view, a bird's-eye view, and a vertical cross-sectional view of a space in which a vehicle 1 in which an MMS (not shown) is mounted is present at a certain point in time.

A laser radar (not shown) is mounted on the rear portion of the vehicle 1. An MMS uses the laser radar to measure the surrounding area of a road R on which the vehicle 1 travels, and acquires data (hereinafter referred to as “position data”) indicating the position of an object that is present in the surrounding area of the road R. The MMS performs measurement in directions (directions in 360 degrees centered about the laser radar) on a plane (hereinafter referred to as a “lateral cross-section”) that is orthogonal to the forward direction of the vehicle 1.

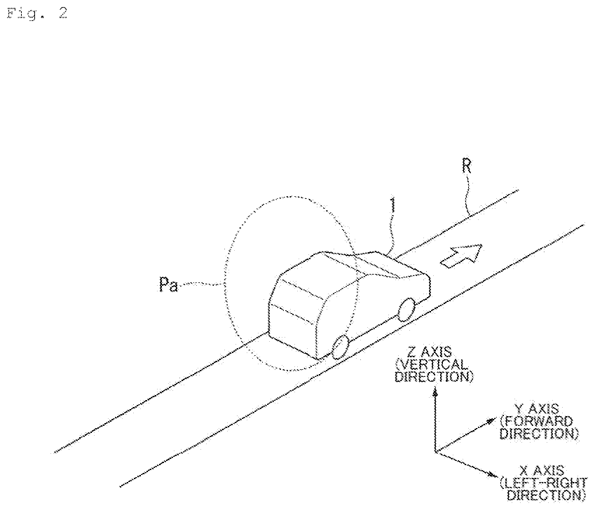

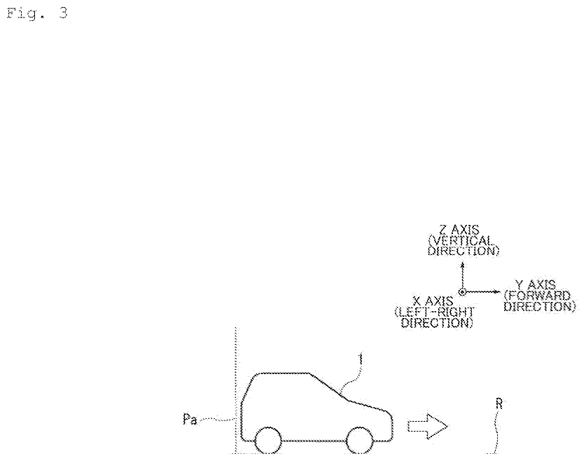

FIGS. 1 to 3 respectively show a position data acquisition position Pa, which is a set of acquisition positions of position data acquired by the laser radar. As shown in FIG. 1, in a plan view, the position data acquisition position Pa is a position on a line extending from the position of the rear portion of the vehicle 1 (i.e., the installation position of the laser radar) in a direction that is orthogonal to the forward direction of the vehicle 1. Also, as shown in FIG. 2, in a bird's-eye view, the position data acquisition position Pa is a position on a circumference of a circle centered about the position of the rear portion of the vehicle 1. Also, as shown in FIG. 3, in a vertical cross-sectional view, the position data acquisition position Pa is a position on a line extending from the position of the rear portion of the vehicle 1 in a direction that is orthogonal to the forward direction of the vehicle 1.

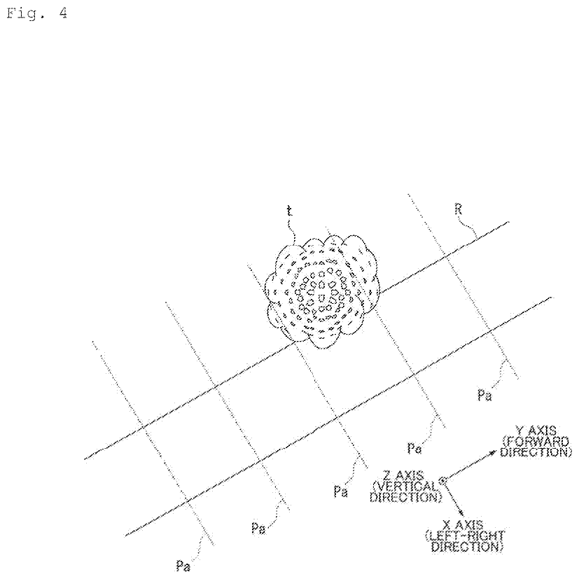

FIGS. 4 to 6 are schematic diagrams showing an example of measurement performed by a conventional MMS. FIGS. 4 to 6 respectively show a plan view, a bird's-eye view, and a vertical cross-sectional view of a space in which the vehicle 1 has traveled, an MMS for measuring the surrounding area of a road R being mounted in the vehicle 1.

The vehicle 1 travels on a road R at a constant speed. Accordingly, the MMS mounted in the vehicle 1 can acquire position data of objects present in the surrounding area of the road R at a certain interval. For example, as shown in FIGS. 4 to 6, one tree t is present on the left side of the road R with respect to the forward direction of the vehicle 1. The MMS acquires the position data of the tree t when passing beside the tree t.

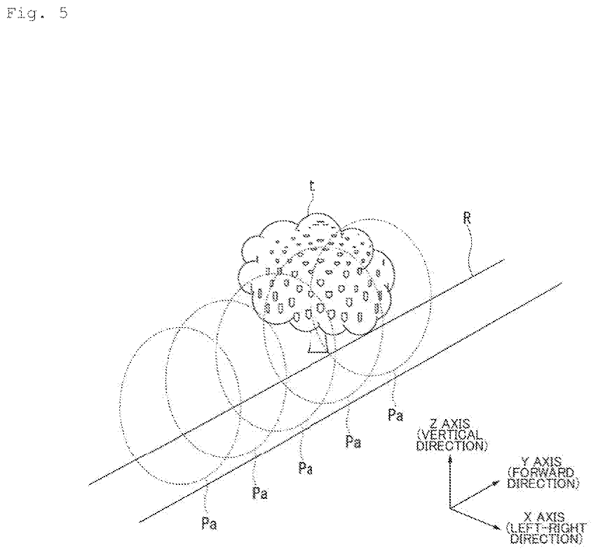

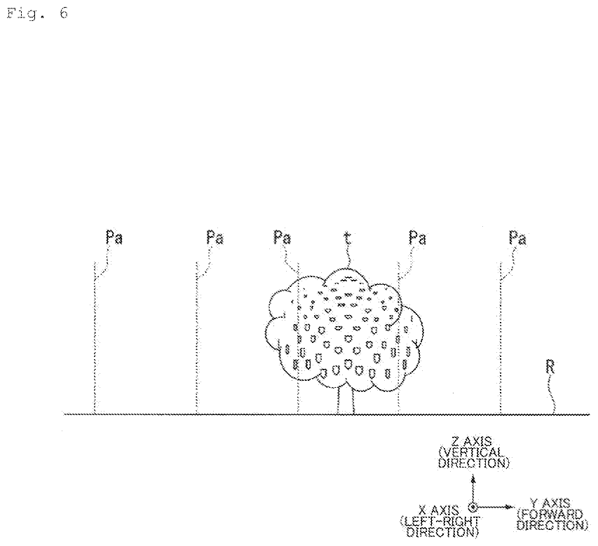

FIGS. 4 to 6 respectively show all position data acquisition positions Pa in the case where the vehicle 1 has traveled on the road R. As shown in FIG. 4, in a plan view, the position data acquisition positions Pa are positions on lines extending in a direction orthogonal to the forward direction of the vehicle 1 and are arranged side by side in parallel at equal intervals. In FIG. 4, two of the lines overlap with the tree t. Also, as shown in FIG. 5, in a bird's-eye view, the position data acquisition positions Pa are positions on circumferences of circles that are arranged side by side at equal intervals in the forward direction of the vehicle 1. Also, as shown in FIG. 6, in a vertical cross-sectional view, the position data acquisition positions Pa are positions of lines that extend in a direction orthogonal to the forward direction of the vehicle 1 and are arranged side by side in parallel at equal intervals. Similarly to FIG. 4, in FIG. 6, two of the lines overlap with the tree t.

Note that in these diagrams, the interval at which position data is acquired (the interval of the position data acquisition positions Pa) is shown exaggerated. In reality, the interval is several centimeters to several tens of centimeters, as with the later-described value. Also, the actual acquisition positions of the position data in the measurement performed by the MMS are, for example, continuous positions instead of discrete positions such as those shown in FIGS. 4 to 6 (i.e., positions such as those on lines arranged side by side at equal intervals, and on circumferences of circles arranged side by side at equal intervals). Specifically, if the actual acquisition positions of the position data in the measurement performed by the MMS are shown in a plan view and a vertical cross-sectional view, the acquisition positions of the position data are positions on curved lines such as sine waves. Also, if the actual acquisition positions of the position data in the measurement performed by the MMS are shown in a bird's eye view, the acquisition positions of the position data are positions on a spiral-shaped line whose central axis is a path through which the laser radar mounted in the vehicle 1 passes.

However, in the following description, in order to facilitate comprehension of the description, it is assumed that the position data acquisition positions Pa in the measurement performed by the MMS are discrete positions such as those in the case where, for example, measurement is performed at equal intervals as in FIGS. 4 to 6. The reason why the position data acquisition positions Pa may be regarded as being discrete positions in this manner will be described simply hereinafter.

For example, the travel speed of the vehicle 1 during measurement performed by the MMS is assumed to be 50 [km/hour] (≈13.9 [m/second]). Also, the number of pieces of position data acquired in one second by the laser radar is assumed to be 30,000 [pieces/second]. Also, the speed at which the laser radar rotates is assumed to be 20 [rotations/second](i.e., 0.05 [seconds/rotation]). In this case, as calculated using the following formula (1), each time the vehicle 1 progresses 69.4 [cm] in the forward direction (y axis direction), the laser radar rotates once in a lateral cross-section (x-z plane).

69.4 [cm]≈13.9 [m/second]÷20 [rotations/second] (1)

Also, as calculated using the following formula (2), each time the laser radar rotates once, 1,500 pieces of position data are acquired. That is, each time the laser radar rotates 0.24 degrees (≈14 minutes, 24 seconds), one piece of position data is obtained.

1,500 [pieces]=30,000 [pieces/second]÷20 [rotations/second] (2)

According to the description above, it can be understood that each of the position data acquisition positions at every 0.24 degrees on a circumference on the x-z plane is present at an equal interval of 69.4 [cm] in the forward direction (y axis direction) of the vehicle 1. Due to the above, the position data acquisition positions Pa can be regarded as being discrete positions that are present at equal intervals in the forward direction (y axis direction) of the vehicle 1.

Note that in the description above, in order to simplify the description, the position data acquisition positions Pa were positions on a circumference of circle in a lateral cross-section (x-z plane). However, in actuality, each of the positions at which the lateral cross-section (x-z plane) coincides with a measurement target is an acquisition position of position data. For this reason, the distance from the laser radar to the acquisition position of the position data differs for each piece of position data.

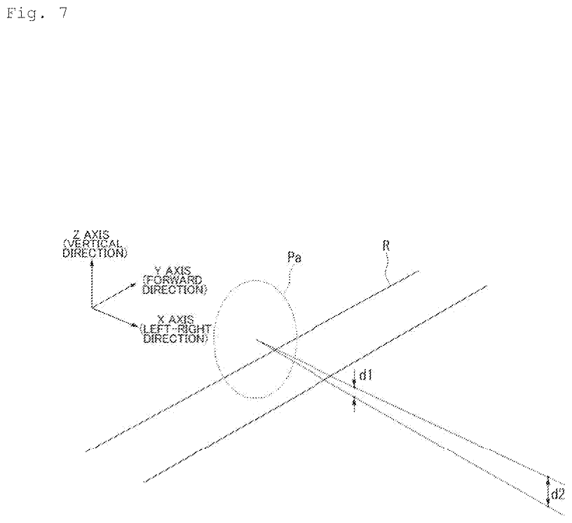

FIG. 7 is a schematic diagram showing a relationship between the distance from the position of the laser radar to the measurement target and the interval of the position data acquisition positions. If the measurement target is present at a position near the position of the laser radar, the interval between two adjacent position data acquisition positions is, for example, an interval such as d1, as shown in FIG. 7. Also, if the measurement target is present at a position far from the position of the laser radar, the interval between two adjacent position data acquisition positions is, for example, an interval such as d2, as shown in FIG. 7. As shown in FIG. 7, the farther the distance from the position of the laser radar to the measurement target is, the wider the interval between two adjacent position data acquisition positions is (i.e., d1<d2).

Specific examples will be given hereinafter. For example, the distance from the laser radar to the measurement target is assumed to be 5 [m]. In this case, the width of the interval between two adjacent position data acquisition positions on a circumference of a circle in a lateral cross-section (x-z plane) is about 1.05 [cm], as shown in formula (3) below.

1.05 [cm]≈5 [m]×100×π÷1,500 [pieces] (3)

For example, the distance from the laser radar to the measurement target is assumed to be 500 [m]. In this case, the width of the interval between two adjacent position data acquisition positions on a circumference of a circle in a lateral cross-section (x-z plane) is about 104.67 [cm], as shown in formula (4) below.

104.67 [cm]×500 [m]×100×π÷1,500 [pieces] (4)

As described above, the longer the distance from the position of the laser radar to the position of the measurement target is, the wider the interval between the two adjacent position data acquisition positions on the circumference of the circle in the lateral cross-section (x-z plane) is.

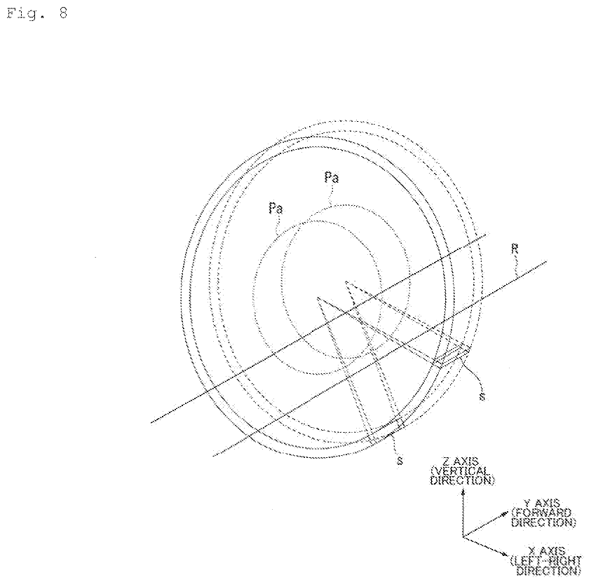

FIG. 8 is a schematic diagram showing the size of a space that can be measured by a conventional MMS. Note that the size of the measurable space in this context represents the size of the smallest measurement target that is reliably measured by the MMS. That is, laser light emitted by the laser radar does not hit a measurement target that is smaller than the size of the measurable space even once, and thus measurement is not performed in some cases.

If the measurement target is at a position near the laser radar, the interval of the two adjacent position data acquisition positions on the circumference of the circle on the lateral cross-section (x-z plane) is relatively narrow compared to the interval of the two adjacent lateral cross-sections (x-z planes). Accordingly, as shown in FIG. 8, the space that can be measured by the conventional MMS is like a cuboid space s that is relatively longer in the forward direction of the vehicle 1.

Note that in actuality, the plane of the space s in the lateral cross-section (x-z plane) has a shape similar to a trapezoid whose side that is farther from the laser radar is slightly longer than the side near the laser radar.

Note that the length of the sides in the depth direction of the space s as viewed from the position of the laser radar is determined according to the resolution of the laser radar.

As shown in FIG. 8, if the distance from the position of the laser radar to the position of the measurement target is the same, the size of the space s is the same, regardless of the height of the position at which the measurement target is present.

In this manner, if the distance from the position of the laser radar to the position of the measurement target is short, the interval of the position data acquisition positions is relatively longer in the forward direction (y axis direction) compared to the circumferential direction. In FIG. 8, the vehicle 1 travels along the road R in a direction from the lower left to the upper right, and the acquisition positions Pa of position data acquired by the MMS during this travel are indicated by two ovals. Also, the two cuboids (spaces s) shown in FIG. 8 are measurable spaces in which the distances from the position of the laser radar to the position of the target object are the same, and the directions (laser light emission directions) on the lateral cross-section (x-z plane) are different from each other.

In this manner, if the distance from the position of the laser radar to the position of the measurement target is short, the space s has a shape like a small cuboid that is longer in the forward direction (y axis direction) of the vehicle 1 and is relatively shorter in the direction in the lateral cross-section (x-z plane).

Here, the lengths of the sides of the spaces s, which are cuboids, correspond to the interval of the adjacent position data acquisition positions in the up, down, front, and rear directions (i.e., the y axis direction and the z axis direction). If the distance from the position of the laser radar to the position of the measurement target is short, the lengths of the sides in the emission direction of the laser light emitted by the laser radar (e.g., the x axis direction in the case where the measurement target is present at the same height as the laser radar) and the sides in the circumferential direction (e.g., the z axis direction in the case where the measurement target is present at the same height as the laser radar) of the circle on the lateral cross-section (x-z plane) are shorter relative to the lengths of the sides in the forward direction (y axis direction) of the vehicle 1. Also, conversely, if the distance from the position of the laser radar to the position of the measurement target is long, the lengths of the sides in the emission direction of the laser light emitted by the laser radar and the sides in the circumferential direction of the circle on the lateral cross-section (x-z plane) are longer relative to the lengths of the sides in the forward direction (y direction) of the vehicle 1.

For example, in the case of the above-described specific example, the lengths of the sides of the space s in the forward direction (y axis direction) of the vehicle 1 are 69.4 [cm]. Also, the lengths of the sides of the space s in the circumferential direction (e.g., the z axis direction if the measurement target is present at the same height as the laser radar) of the circle in the lateral cross-section (x-z plane) are 1.05 [cm]. Also, if the resolution of the laser radar is, for example, 5 [cm], the lengths of the sides of the space s in the emission direction of the laser light emitted by the laser radar (e.g., the x axis direction if the measurement target is present at the same height as the laser radar) are 5 [cm]. Accordingly, the space s has the shape of a 69.4 [cm]×1.05 [cm]×5 [cm] cuboid.

On the other hand, the space s in the case where the distance from the laser radar to the measurement target is long has the shape of a cuboid that is relatively shorter in the forward direction of the vehicle 1, although this is not illustrated in the drawings. For example, in the case of the above-described specific example, the lengths of the sides of the space s in the forward direction (y axis direction) of the vehicle 1 are 69.4 [cm]. Also, the lengths of the sides of the space s in the circumferential direction (e.g., the z axis direction if the measurement target is present at the same height as the laser radar) of the circle on the lateral cross-section (x-z plane) are 104.67 [cm]. Also, if the resolution of the laser radar is, for example, 100 [cm], the lengths of the sides of the space s in the emission direction of the laser light emitted by the laser radar (e.g., the x axis direction if the measurement target is present at the same height as the laser radar) are 100 [cm]. Accordingly, the space s has the shape of a 69.4 [cm]×104.67 [cm]×100 [cm] cuboid.

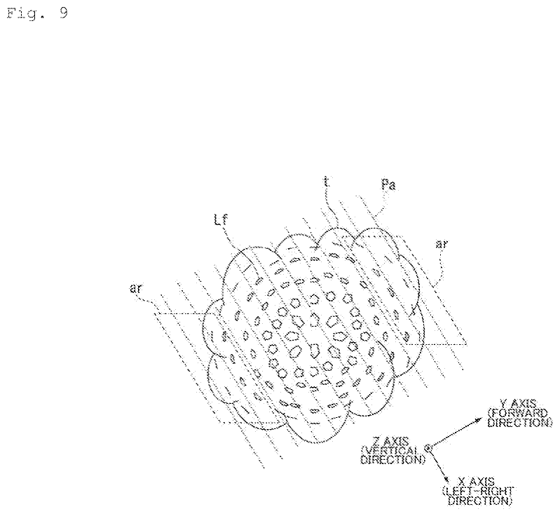

FIGS. 9 and 10 are schematic diagrams showing measurement-undetected regions in the case where the measurement target is a tree. FIGS. 9 and 10 respectively show a plan view and a vertical cross-sectional view of a periphery of a tree t that is present in a space through which the vehicle 1 travels, an MMS for measuring the surrounding area of a road R being mounted in the vehicle 1.

As shown in FIGS. 9 and 10, many leaves Lf whose position data is to be acquired are growing on the tree t. Also, FIGS. 9 and 10 show the position data acquisition positions Pa. As shown in FIGS. 9 and 10, in the tree t, measurement-undetected regions ar occur on the front side and the rear side in the forward direction (y axis direction) of the vehicle 1. The measurement-undetected regions ar are regions in which measurement performed by the MMS is difficult.

The reason why the measurement-undetected regions ar occur is because, as shown in FIG. 9, in generally, the orientations of the front surfaces of the leaves Lf radially face a direction on the outer side of the tree t with the trunk of the tree t behind them in order to intake sunlight. Due to this, leaves Lf growing at positions in the forward direction (y axis direction) of the vehicle 1 and in a direction opposite thereto on the tree t have side surfaces facing the vehicle 1 passing through the vicinity. In general, the length of the side surface of a leaf Lf (i.e., the thickness of the leaf Lf) is often thin (e.g., less than 1 [mm]). Also, in general, it is difficult for the MMS to acquire position data at an interval narrower than the thickness of the leaf Lf.

If the measurement-undetected region ar occurs, the shape of the tree t obtained based on the position data acquired by the MMS becomes a shape that is thinner in the forward direction (y axis direction) of the vehicle 1 compared to the actual shape of the tree t. In this manner, if the measurement-undetected region ar occurs, there is a risk that misrecognition will occur in which the MMS recognizes the size of the measurement target as being smaller than it is in reality.

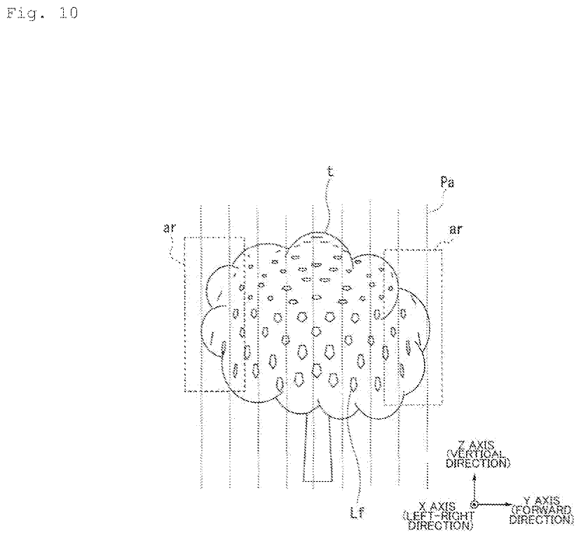

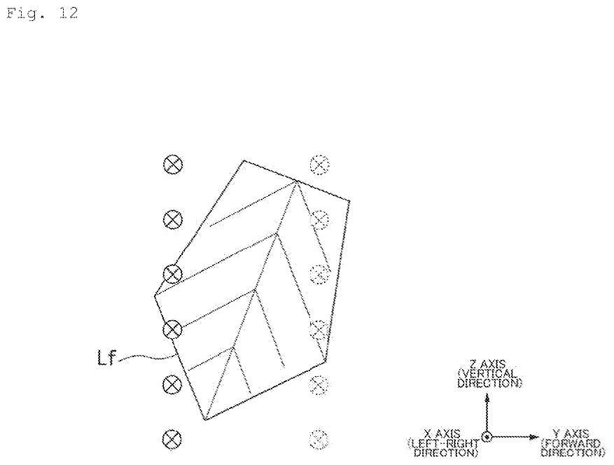

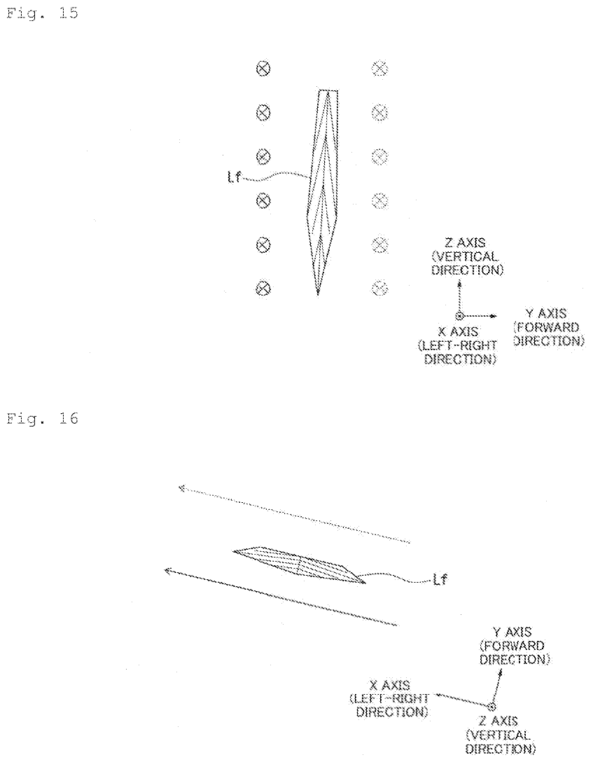

FIGS. 11 to 16 are schematic diagrams showing a relationship between the orientation of the leaf Lf and the emission direction of the laser light emitted by the laser radar. In FIGS. 11, 13, 14, and 16, the solid line arrows and the dotted-line arrows indicate emission of the laser light. The solid-line arrows and the dotted-line arrows are distinguished by the difference in the acquisition timing of the position data. That is, the emission of the laser light to the position data acquisition positions Pa at a given acquisition timing is indicated by the solid-line arrows. On the other hand, the emission of the laser light to the position data acquisition positions Pa at an acquisition timing different from the above-described acquisition timing is indicated by the dotted-line arrows.

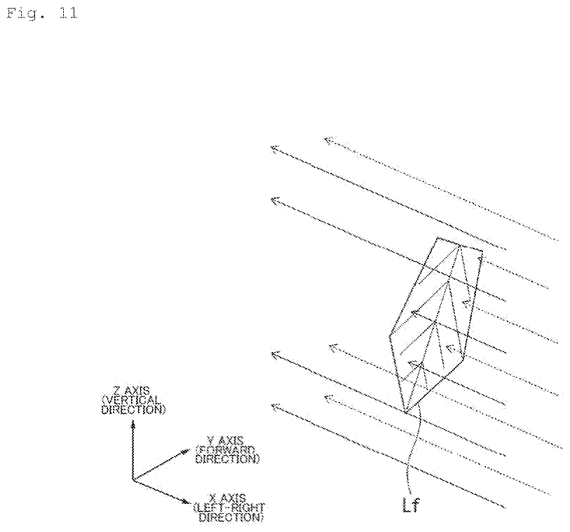

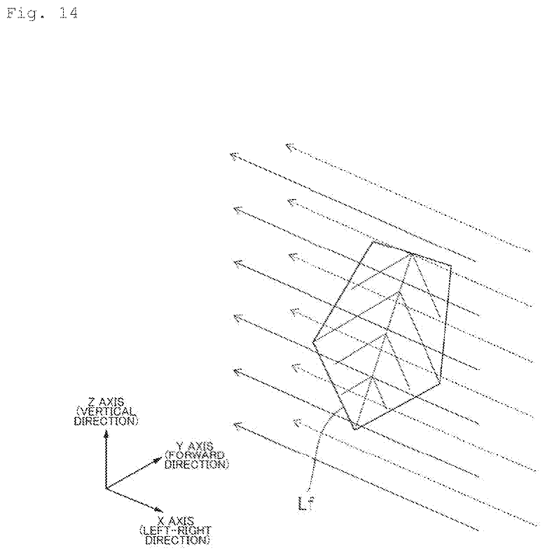

Also, the marks in which an X is drawn in a circle in FIGS. 12 and 15 correspond to the arrows in FIGS. 11, 13, 14, and 16. The marks indicate that the laser light has been emitted in the depth direction from the near direction of the drawing plane. The marks drawn with solid lines and the marks drawn with dotted lines are distinguished by the difference in the acquisition timing of the position data. That is, the emission of the laser light to the position data acquisition positions Pa at a given acquisition timing is indicated by the solid-line marks. On the other hand, the emission of the laser light to the position data acquisition positions Pa at an acquisition timing different from the above-described acquisition timing is indicated by the dotted-line marks.

FIGS. 11 to 13 respectively show a bird's-eye view, a vertical cross-sectional view, and a plan view of the periphery of a leaf Lf growing at a position that is not the measurement-undetected region ar on the tree t that is present in the space through which the vehicle 1 is traveling, an MMS for measuring the surrounding area of a road R being mounted in the vehicle 1.

As shown in FIGS. 11 to 13, the front surface of the leaf Lf growing at the position that is not the measurement-undetected region ar faces a direction that is nearly parallel to the y-z plane. That is, the front surface of the leaf Lf faces a direction that is nearly orthogonal to the emission direction (x axis direction) of the laser light emitted by the laser radar. According to this, the leaf Lf growing at the position that is not the measurement-undetected region ar is easily hit by the laser light emitted from the laser radar.

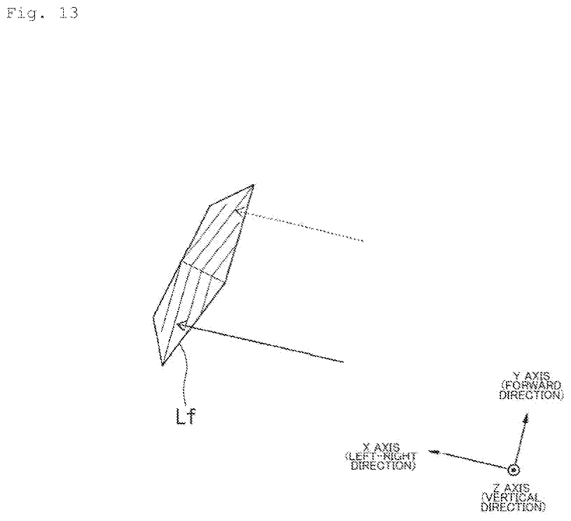

On the other hand, FIGS. 14 to 16 respectively show a bird's-eye view, a vertical cross-sectional view, and a plan view of the periphery of a leaf Lf growing in the measurement-undetected region ar on the tree t that is present in the space through which the vehicle 1 is traveling, an MMS for measuring the surrounding area of a road R being mounted in the vehicle 1.

As shown in FIGS. 14 to 16, the front surface of the leaf Lf growing in the measurement-undetected region ar faces a direction that is nearly parallel to the lateral cross-section (x-z plane). That is, the front surface of the leaf Lf faces a direction that is nearly parallel to the emission direction (x axis direction) of the laser light emitted by the laser radar. According to this, the leaf Lf growing in the measurement-undetected region ar is not easily hit by the laser light emitted from the laser radar.

Also, the direction in which the interval at which the laser light is emitted by the laser radar is short is the z axis direction, whereas as shown in FIG. 15, the longitudinal direction of the leaf Lf is approximately the z axis direction, and therefore it can be said that the laser light emitted from the laser radar is not likely to hit even if the leaf Lf is slightly inclined.

In this manner, due to the relationship between the orientation of the leaf Lf and the emission direction of the laser light emitted by the laser radar, the measurement-undetected regions ar occur in the frontward portion and the rearward portion in the y axis direction on the tree t (i.e., the portion of the tree t on the forward direction side and the portion of the tree t on the reverse direction side of the vehicle 1). The reason why the measurement-undetected regions ar occur is because no component in the y axis direction (forward direction of the vehicle 1) is included in the emission direction of the laser light emitted by the laser radar.

That is, the front surfaces of the leaves Lf growing on the frontward portion and the rearward portion in the y axis direction on the tree t (i.e., the portion of the tree t on the forward direction side and the portion of the tree t on the opposite direction side of the vehicle 1) often face the forward direction or the opposite direction of the vehicle 1. Accordingly, the leaves Lf growing on the frontward portion and the rearward portion in the y axis direction on the tree t grow such that their side surfaces face the vehicle 1. On the other hand, the laser light is emitted in a direction orthogonal to the forward direction of the vehicle 1 (i.e., the direction in which the component in the y axis direction is not included). Accordingly, the mutual positional relationship between front surface of the leaf Lf and the emission direction of the laser light is parallel, and therefore the measurement-undetected region ar occurs.

Due to the measurement-undetected regions ar occurring on the frontward portion and the rearward portion in the y axis direction, there is a possibility that misrecognition will occur in which the MMS recognizes the width of the tree t as being thinner than it is in reality.

### First Embodiment

Hereinafter, a first embodiment of the present invention will be described with reference to the drawings.

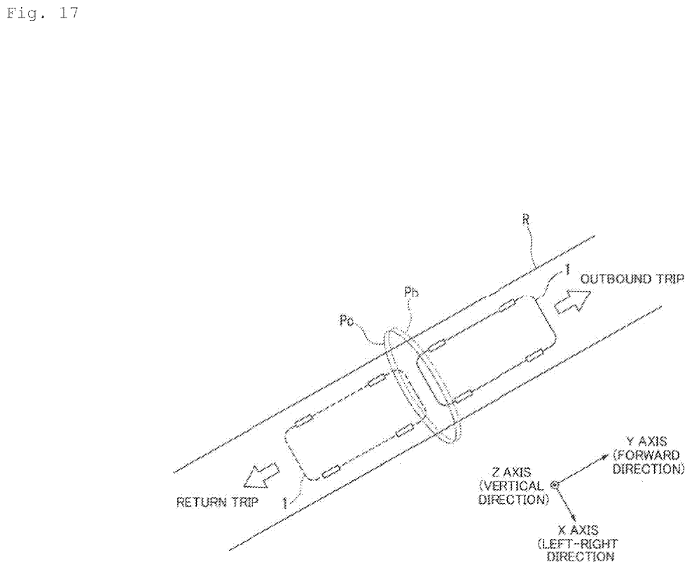

FIGS. 17 to 19 are schematic diagrams showing acquisition positions of position data acquired by an MMS according to a first embodiment of the present invention. In the present embodiment, a vehicle 1 travels round-trip on a road R. FIGS. 17 to 19 respectively show a plan view, a bird's-eye view, and a vertical cross-sectional view of a space in which the vehicle 1 in which the MMS is mounted is present, at one point in time on an outbound trip and at one point in time on a return trip.

A laser radar (not shown) is mounted on the rear portion of the vehicle 1. The MMS measures the surrounding area of the road R on which the vehicle 1 travels using a laser radar, and acquires position data for an object that is present in the surrounding area of the road R.

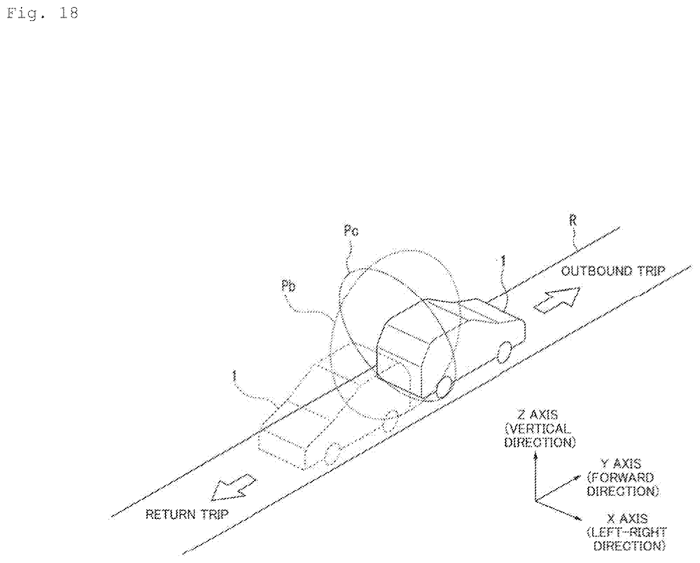

However, the vehicle 1 according to the present embodiment differs from the above-described conventional technique in that, as can be understood by referencing FIG. 19 in particular, the plane formed by the position data acquisition positions has a depression angle with respect to the lateral cross-section (which is a plane that is orthogonal to the forward direction of the vehicle 1). Hereinafter, the plane having this depression angle will be referred to as a “measurement plane”. The MMS performs measurement in each direction on the measurement plane and acquires position data. Note that the measurement plane may also have an elevation angle instead of a depression angle. However, with a depression angle, the measurement plane is not blocked by the vehicle 1 when measuring near the road surface, which is more advantageous.

In FIGS. 17 to 19, the vehicle 1 on the outbound trip is indicated by a solid line, and the vehicle 1 on the return trip is indicated by a broken line. Also, in FIGS. 17 to 19, the position data acquisition position Pb, which is a set of acquisition positions of the position data acquired by the MMS on the outbound trip, is indicated by a dotted line. Also, in FIGS. 17 to 19, the position data acquisition position Pc, which is a set of acquisition positions of the position data acquired by the MMS on the return trip, are indicated by a one-dot chain line.

As can be understood from the vertical cross-sectional view in FIG. 19, the measurement plane formed by the position data acquisition position Pb on the outbound trip has a depression angle with respect to the lateral cross-section taken with respect to the outbound trip direction. On the other hand, the measurement plane formed by the position data acquisition position Pc on the return trip has a depression angle with respect to the lateral cross-section taken with respect to the return trip direction. In other words, the measurement plane formed by the position data acquisition position Pc on the return trip has an elevation angle with respect to the lateral cross-section taken with respect to the outbound trip direction. Accordingly, as can be understood from the vertical cross-sectional view in FIG. 19, the measurement plane on the outbound trip and the measurement plane on the return trip intersect each other.

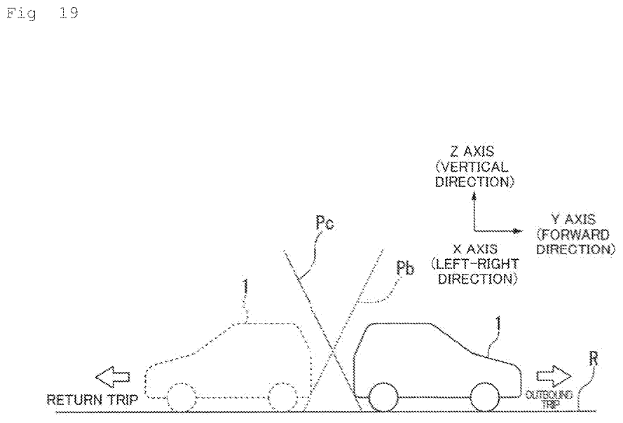

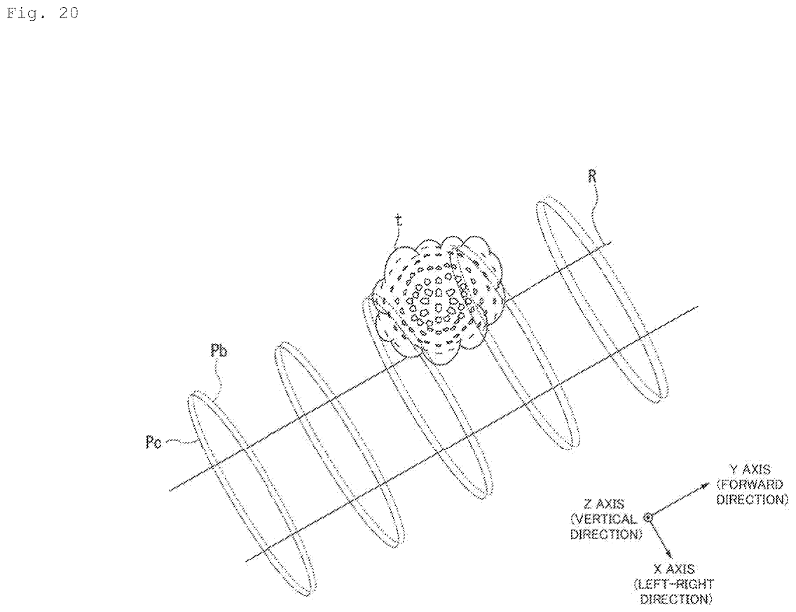

FIGS. 20 to 22 are schematic diagrams showing an example of measurement performed by the MMS according to the first embodiment of the present invention. FIGS. 20 to 22 respectively show a plan view, a bird's-eye view, and an orthogonal cross-sectional view of a space in which the vehicle 1 has traveled round-trip, an MMS for measuring the surrounding area of the road R being mounted in the vehicle 1.

The vehicle 1 travels round-trip on the road R at a constant speed. Accordingly, the MMS mounted in the vehicle 1 can acquire the position data of the object present in the surrounding area of the road R at a certain interval on the outbound trip and the return trip. For example, as shown in FIGS. 20 to 22, one tree t is present on the left side of the road R with respect to the outbound trip direction of the vehicle 1 (i.e., the left side of the road R with respect to the return trip direction of the vehicle 1). When passing through beside the tree t, the MMS acquires the position data of the tree t on the outbound trip and the return trip.

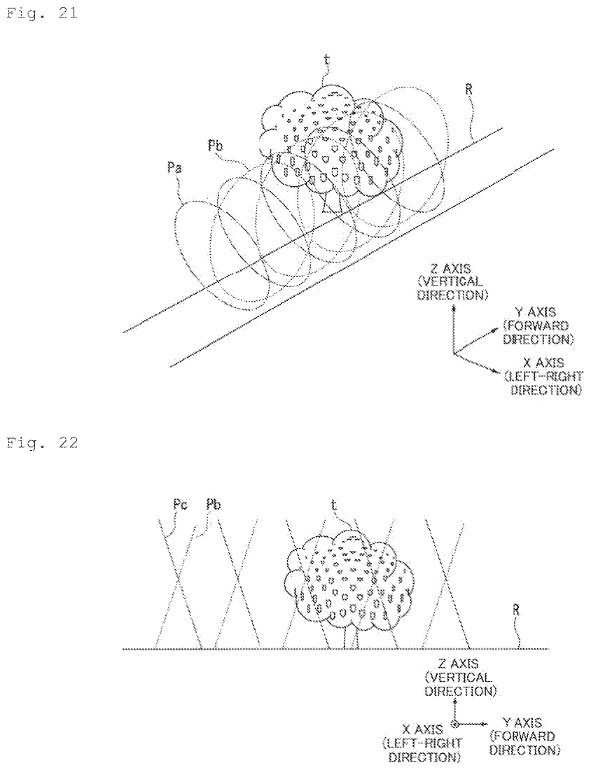

FIGS. 20 to 22 respectively show all of the position data acquisition positions Pb in the case where the vehicle 1 has performed outbound-trip travel on the road R and all of the position data acquisition positions Pc in the case where the vehicle 1 has performed return-trip travel on the road R. As can be understood from the plan view in FIG. 20 and the vertical cross-sectional view in FIG. 22, two each of the measurement planes formed by the position data acquisition positions Pb on the outbound trip and the measurement planes formed by the position data acquisition positions Pc on the return trip overlap with the tree t.

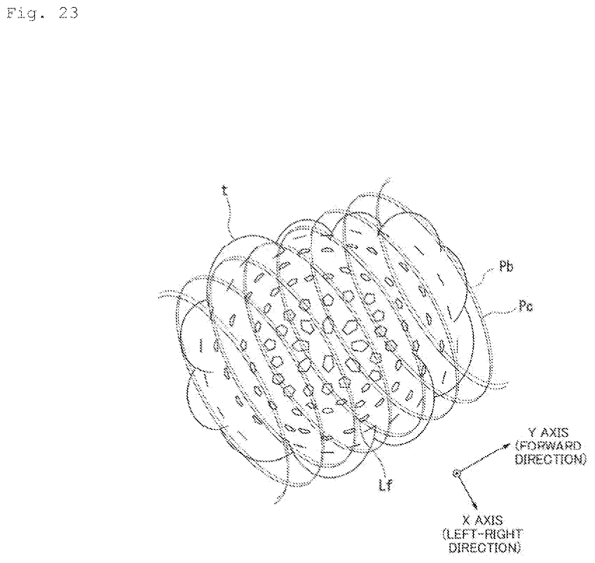

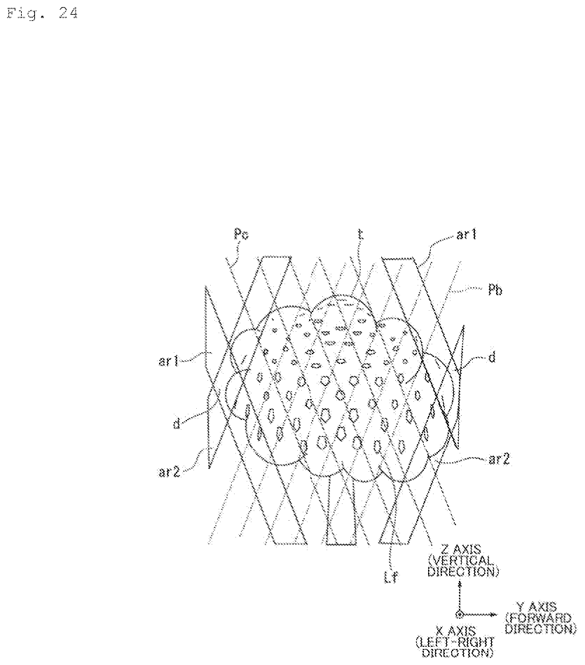

FIGS. 23 and 24 are schematic diagrams for illustrating measurement-undetected regions in the case where the measurement target is a tree. FIGS. 23 and 24 respectively show a plan view and a vertical cross-sectional view of the periphery of the tree t that is present in a space through which the vehicle 1 has traveled round-trip, the MMS for measuring the surrounding area of the road R being mounted in the vehicle 1.

As shown in FIGS. 23 and 24, many leaves Lf whose position data is to be acquired grow on the tree t. Also, in FIGS. 23 and 24, the position data acquisition positions Pb on the outbound trip and the position data acquisition positions Pc on the return trip are shown. As shown in FIG. 24, on the tree t, the measurement-undetected regions occur on the forward direction side and the opposite direction side of the vehicle 1 (the frontward side and the rearward side in the y axis direction). As described above, the measurement-undetected regions are regions in which measurement performed by the MMS is difficult. Note that the measurement-undetected regions are omitted in FIG. 23 so that the drawing does not become complicated.

As shown in FIG. 24, the measurement-undetected regions ar1 are measurement-undetected regions that occur when the position data of the outbound trip is acquired. On the other hand, the measurement-undetected regions ar2 are measurement-undetected regions that occur when the position data of the return trip is acquired. Two each of the measurement-undetected regions ar1 and the measurement-undetected regions ar2 are present, since they occur on the forward direction side and the opposite direction side of the vehicle 1 (the frontward side and the rearward side in the y axis direction) on the tree t.

Also, as shown in FIG. 24, the measurement-undetected regions ar1 and the measurement-undetected regions ar2 respectively have the same depression angle (inclination) as the measurement plane on the outbound trip and the measurement plane on the return trip, and therefore intersect each other. Accordingly, as shown in FIG. 24, on the tree t, overlapping regions d occur on the forward direction side and the opposite direction side of the vehicle 1 (the frontward side and the rearward side in the y axis direction).

The ranges of the measurement-undetected regions ar1 that are not the overlapping regions d are the regions that are not the measurement-undetected regions ar2. Accordingly, these regions are regions in which it is difficult to acquire the position data on the outbound trip, but are regions in which it is possible to acquire the position data on the return trip. On the other hand, the ranges of the measurement-undetected regions ar2 that are not the overlapping regions d are regions that are not the measurement-undetected regions ar1. Accordingly, these regions are regions in which it is difficult to acquire the position data on the return trip, but are regions in which it is possible to acquire the position data on the outbound trip.

In this manner, according to the present embodiment, the measurement performed by the MMS on the outbound trip and the measurement performed by the MMS on the return trip complement each other, and therefore the regions in which it is difficult to acquire the position data on both the outbound trip and the return trip are limited to only the overlapping regions d. Due to the plane that has a depression angle (or an elevation angle) with respect to the lateral cross-section, which is a plane that is orthogonal to the forward direction of the vehicle 1, being the measurement plane and the vehicle 1 traveling round-trip, for example, as shown in FIG. 24, the measurement-undetected regions are significantly reduced compared to the conventional technique. Accordingly, the likelihood that misrecognition will occur in which the MMS recognizes the width of the tree t as being thinner than it is in reality due to the occurrence of the measurement-undetected regions ar is significantly reduced compared to the conventional technique.

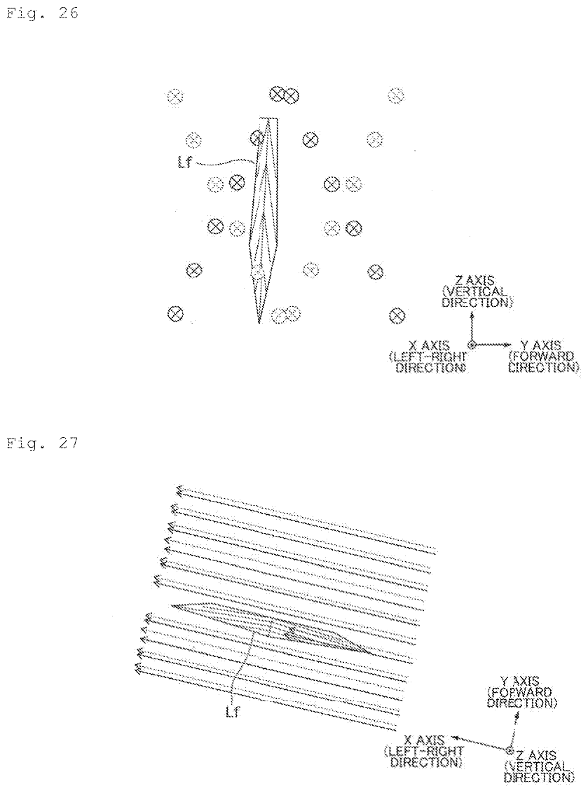

FIGS. 25 to 27 are schematic diagrams showing a relationship between the orientation of the leaf Lf and the emission direction of the laser light emitted by the laser radar. In FIGS. 25 and 27, the solid-line arrows and the dotted-line arrows indicate the emission of the laser light. The solid-line arrows and the dotted-line arrows are distinguished by the difference in the acquisition timing of the position data. That is, the emission of the laser light to the position data acquisition positions Pb (or the position data acquisition positions Pc) at a given acquisition timing is indicated by the solid-line arrows. On the other hand, the emission of the laser light to the position data acquisition positions Pb (or the position data acquisition positions Pc) at an acquisition timing different from the above-described acquisition timing is indicated by the dotted-line arrows.

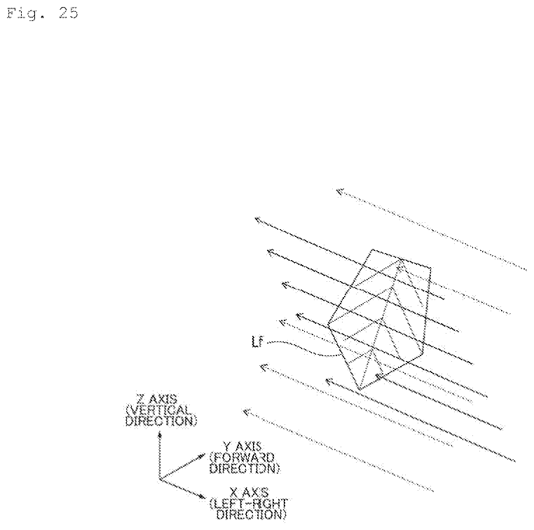

Also, the marks in which an X is drawn in a circle in FIG. 26 correspond to the arrows in FIGS. 25 and 27. The mark indicates that the laser light has been emitted in the depth direction from the near direction of the drawing. As shown in FIG. 26, the marks include marks that are arranged side by side in two rows from the upper left to the lower right of the drawing, and marks that are arranged side by side in two rows from the upper right to the lower left of the drawing. The marks that are arranged side by side from the upper left to the lower right of the drawing indicate laser lights emitted when the vehicle 1 travels on the outbound trip. On the other hand, the marks arranged side by side from the upper right to the lower left of the drawing indicate laser lights emitted when the vehicle 1 travels on the return trip.

The marks drawn with solid lines and the marks drawn with dotted lines are distinguished by the difference in the acquisition timing of the position data. That is, the emission of the laser light to the position data acquisition positions Pb (or the position data acquisition positions Pc) at a given acquisition timing is indicated by the solid-line marks. On the other hand, the emission of the laser light to the position data acquisition positions Pb (or the position data acquisition positions Pc) at an acquisition timing different from the above-described acquisition timing is indicated by the dotted-line marks.

FIGS. 25 to 27 respectively show a bird's-eye view, a vertical cross-sectional view, and a plan view of the periphery of the leaf Lf growing on the tree t that is present in the space through which the vehicle 1 travels round-trip, an MMS for measuring the surrounding area of the road R being mounted in the vehicle 1. Note that the orientation of the front surface of the leaf Lf shown in FIGS. 25 to 27 is the same as the orientation of the front surface of leaf Lf shown in FIGS. 14 to 16.

In FIGS. 14 to 16, the leaf Lf was in a state in which laser light emitted from the laser radar was not likely to hit. In contrast to this, according to the present embodiment, as shown in FIGS. 25 to 27, on the outbound trip and the return trip, measurement of the measurement planes that are different from each other is performed, and therefore the likelihood that the laser light will hit the leaf Lf increases even with the orientation of the leaf Lf that was not likely to be hit by the laser light emitted by the laser radar in the conventional technique. This makes it possible to acquire the position data of the leaf Lf that could not be acquired with the conventional technique.

Note that according to the present embodiment, if the position of the leaf Lf is at a different height than the position of the laser radar in the case where the orientation of the front surface of the leaf Lf is the orientation shown in FIGS. 25 to 27, the likelihood that the laser light will hit the leaf Lf increases. This is because due to the measurement plane having a depression angle (or elevation angle), a component in the forward direction or the opposite direction of the vehicle 1 (frontward direction or rearward direction in the y axis direction) is included in the emission direction of the laser light, with respect to the direction different from the height of the position of the laser radar.

Also, the greater the difference between the height of the position of the leaf Lf and the height of the position of the laser radar is, the more the component in the forward direction or the opposite direction of the vehicle 1 (the frontward direction or the rearward direction in the y axis direction), which is included in the emission direction of the laser light, is included, and therefore the likelihood that the laser light will hit the leaf Lf is greater.

For example, if the laser radar is mounted on the roof of the vehicle 1, the position data acquisition positions Pb or PC acquired by the MMS are located on the measurement plane having a depression angle (or an elevation angle) in the forward direction (y axis direction) instead of on the lateral cross-section (x-z plane), which is a plane that is orthogonal to the forward direction (y axis direction) of the vehicle 1. Accordingly, the emission direction of the laser light emitted to the measurement target located at a position higher than the height of the roof of the vehicle position (i.e., the installation height of the laser radar) includes a component in the opposite direction (the plus direction in the y axis direction) of the forward direction of the vehicle 1. On the other hand, the emission direction of the laser light emitted to the measurement target located at a position lower than the height of the roof of the vehicle position includes a component in the forward direction of the vehicle 1 (the minus direction in the y axis direction).

Specifically, if the laser light is emitted to a position at the same height as the position of the laser radar, the laser light is emitted in a direction (X axis direction) that is orthogonal to the forward direction of the vehicle 1. In contrast to this, if the laser light is emitted to a position that is higher than the position of the laser radar, the laser light is emitted in a direction that is obliquely upward and frontward of the vehicle 1 (if the measurement plane has a depression angle). Also, if the laser light is emitted to a position that is lower than the position of the laser radar, the laser light is emitted in a direction that is obliquely downward and rearward of the vehicle 1 (if the measurement plane has a depression angle). Accordingly, even if the leaf Lf is growing with its side surface facing the vehicle 1, if the heights of the position of the leaf Lf and the position of the laser radar are different from each other, the laser light is emitted in a direction including a component in the frontward direction or the rearward direction of the vehicle 1, and therefore the likelihood that the laser light will hit increases.

Operation of MMS

Hereinafter, an example of operations of the MMS (measurement apparatus) will be described.

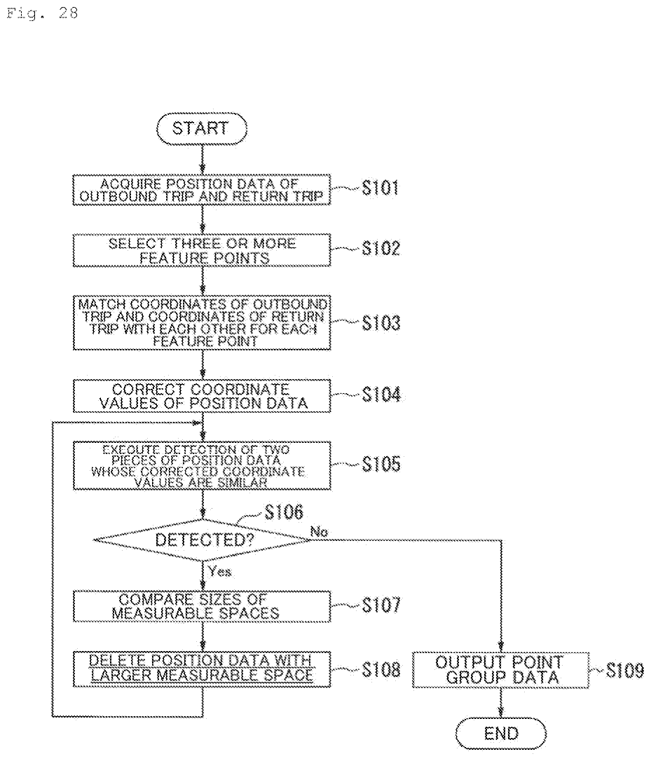

FIG. 28 is a flowchart showing operations of an MMS according to a first embodiment of the present invention.

The MMS acquires the position data obtained by the laser radar during outbound-trip travel of the vehicle 1 and the position data obtained by the laser radar during return-trip travel of the vehicle 1 from an external apparatus or the like that includes, for example, a laser radar (step S101). That is, the MMS acquires the above-described position data of the position data acquisition positions Pb and the position data of the position data acquisition positions Pc.

The MMS selects three or more feature points near the travel route (step S102). The feature points are, for example, a corner of a roof of a specific building, a trunk of a specific tree, or the like. Note that it is desirable that two of the feature points to be selected are respectively an object that is present near the departure point of the outbound trip on the travel route of the vehicle 1, and an object that is present near the departure point of the return trip on the travel route of the vehicle 1 (i.e., near the arrival point of the outbound trip). Note that the purpose of this is to increase the precision of later-described correction processing of coordinates.

The MMS extracts the position data corresponding to the feature points from the position data obtained on the outbound trip. Also, the MMS extracts the position data corresponding to the feature points from the position data obtained on the return trip. For each feature point, the MMS performs coordinate alignment processing for matching the coordinates indicated by the position data obtained on the outbound trip (hereinafter referred to as “outbound trip coordinates”) and the coordinates indicated by the position data obtained on the return trip (hereinafter referred to as “return trip coordinates”) (step S103).

Specifically, for example, the MMS reflects the difference values obtained by performing adjustment at this time in the return trip coordinates of the other feature points so as to cause the coordinate values of the return trip coordinates to match the coordinate values of the outbound trip coordinates for one of the feature points. Then, the MMS performs adjustment such that the coordinate values of the outbound trip coordinates and the coordinate values of the return trip coordinates are closer to each other by increasing and decreasing the values of the other return trip coordinates while maintaining the ratio of the coordinate values between the feature points.

Furthermore, the MMS corrects the coordinate values of the coordinates indicated by the position data other than the feature points based on the coordinate values of the coordinates indicated by the position data of the feature points subjected to coordinate alignment processing (step S104).

Next, the MMS executes detection processing for detecting a set of two pieces of position data for which the corrected coordinate values are similar to each other in the position data of the outbound trip and the position data of the return trip (step S105). Note that the determination of whether or not the corrected coordinate values are similar to each other is performed based on, for example, whether or not the distance between the coordinates indicated by two pieces of position data is less than the interval of the resolution of the laser radar.

If two pieces of position data whose corrected coordinate values are similar to each other are detected in the position data of the outbound trip and the position data of the return trip (step S106: Yes), the MMS compares the sizes of the spaces that can be measured by the MMS in the two pieces of position data whose corrected coordinate values are similar to each other (i.e., the above-described sizes of the smallest measurement targets for which measurement is performed reliably by the MMS) (step S107).

The MMS deletes the position data for which the size of the measurable space is greater out of the two pieces of position data based on the result of the above-described comparison processing (step S108). Note that the size of the space that can be measured by the MMS changes depending on the distance between the position of the laser radar and the position of the measurement target, the travel speed of the vehicle 1, and the like, as described above.

Note that in the above-described comparison processing, the MMS may also, for example, leave the coordinates corresponding to the middle of the coordinates indicated by the two pieces of position data and delete the above-described two pieces of position data instead of leaving the position data for which the size of the measurable space is smaller and deleting the position data for which the size of the measurable space is greater out of the two pieces of position data.

The MMS repeatedly executes the processing from step S105 to step S108 described above until the two pieces of position data for which the corrected coordinate values are similar to each other are no longer detected. If two pieces of position data whose corrected coordinate values are similar to each other are not detected (step S106: No), the MMS outputs point group data, which is a set of pieces of position data subjected to the above-described processing to an apparatus (e.g., an external apparatus or the like) for performing subsequent processing of the MMS (step S109). With that, the processing of the MMS indicated by the flowchart in FIG. 28 ends.

Note that due to the above-described processing, the two pieces of position data whose corrected coordinate values are similar to each other are replaced with one piece of position data in the point group data of the outbound trip and the point group data of the return trip, and therefore the apparatus for performing the subsequent processing can use the point group data output from the MMS as general point group data.

Coordinate Alignment Processing

Hereinafter, the above-described coordinate alignment processing will be described in further detail.

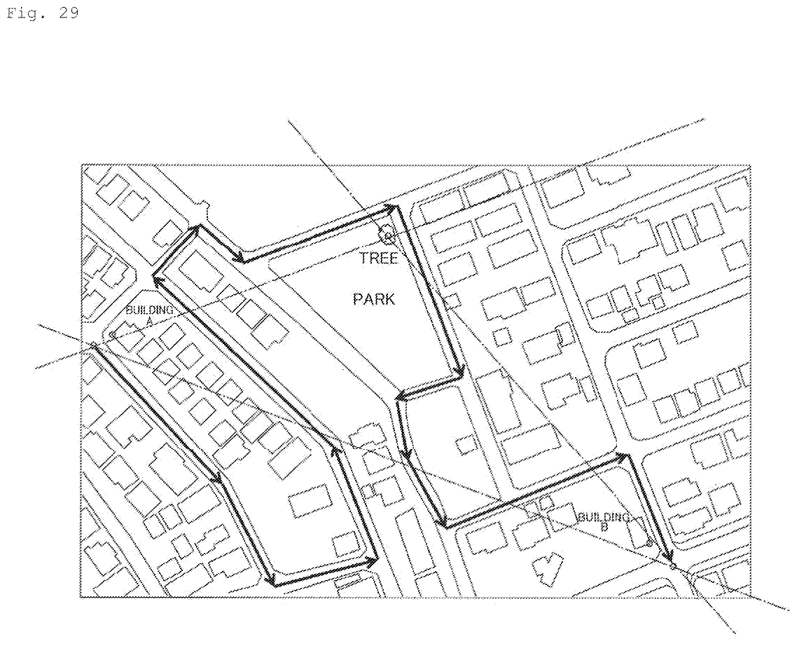

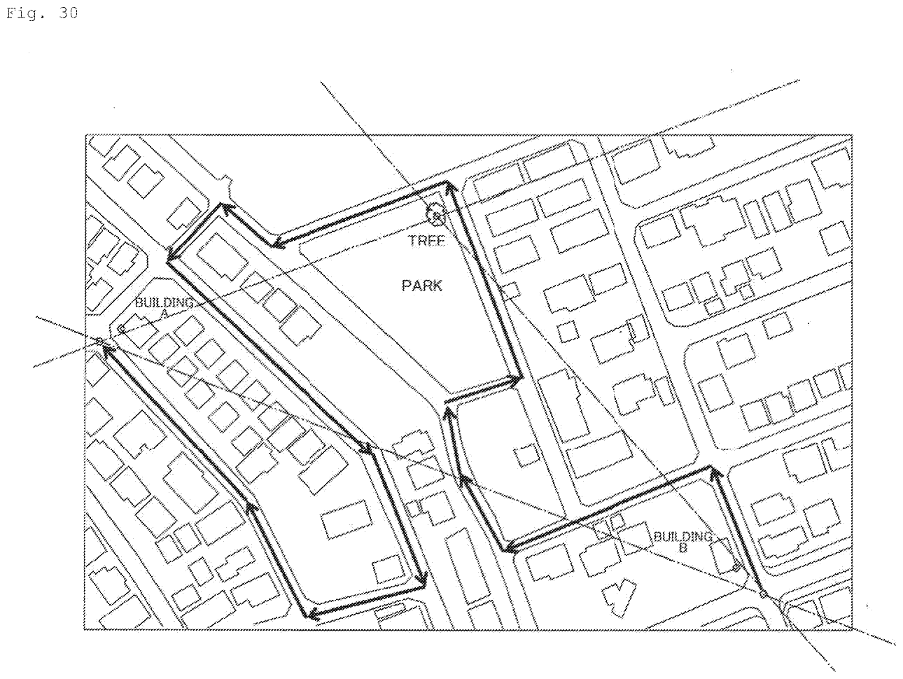

FIGS. 29 and 30 are schematic diagrams for illustrating coordinate alignment processing performed by the MMS according to the first embodiment of the present invention.

FIG. 29 shows the travel route of the outbound trip of the vehicle 1 and three selected feature points on a map. On the other hand, FIG. 30 shows the travel route of the return trip of the vehicle 1 and three selected feature points on a map.

On the travel route of the outbound trip shown in FIG. 29, the first feature point is a portion of a corner of a building A that is present near the departure point of the outbound trip of the vehicle 1. Also, the second feature point is a trunk of a tree that is present in a park on the travel route. Also, the third feature point is a portion of a corner of a building B that is present near the arrival point of the outbound trip of the vehicle 1 (i.e., the departure point of the return trip).

On the other hand, on the travel route of the return trip shown in FIG. 30, the first feature point is a portion of a corner of the building B that is present near the departure point of the return trip of the vehicle 1. Also, the second feature point is a trunk of a tree that is present in a park on the travel route. Also, the third feature point is a portion of a corner of a building A that is present near the arrival point of the return trip of the vehicle 1 (i.e., the departure point of the outbound trip).

For the coordinates indicated by the position data corresponding to the feature points on the outbound trip and the coordinates indicated by the position data corresponding to the feature points on the return trip, the MMS matches the coordinate value of one coordinate with the coordinate value of another coordinate, and thus overlays the position data of the feature points on the outbound trip and the position data of the feature points on the return trip on each other. That is, the MMS overlays the position data corresponding to the “corner of building A” on the outbound trip and the position data corresponding to the “corner of building A” on the return trip on each other. Also, the MMS overlays the position data corresponding to the “trunk of a tree in the park” on the outbound trip and the position data corresponding to the “trunk of a tree in the park” on the return trip on each other. Also, the MMS overlays the position data corresponding to the “corner of building B” on the outbound trip and the position data corresponding to the “corner of building B” on the return trip on each other.

The MMS overlays the position data corresponding to the same feature points to correct the coordinate values of the coordinates indicated by the position data. Then, the MMS furthermore corrects the coordinate values of the coordinates indicated by all of the other position data based on this correction.

Note that the reason why the MMS performs coordinate alignment processing such as that described above is as follows. In general, an MMS that acquires position data measures positions using a satellite positioning system such as a GPS (Global Positioning System). However, an error is included in the position measured by the GPS or the like in some cases. In order to eliminate such an error, the MMS performs coordinate alignment processing for aligning position data of the above-described feature points to overlay point group data on each other, and performs correction of the coordinate values of the coordinates indicated by all of the position data (point group data).

Note that the reason why three or more feature points are used is as follows. In general, the position data acquired by the MMS is three-dimensional information, and is distributed widely near the travel route of the vehicle 1 on a map. For example, the height of an object such as a building in the periphery is about several meters to several tens of meters with respect to a range of the periphery of the travel route, whose width is about several km² to several tens of km². That is, although the elevation of the geography is added, the space of the range that is the measurement target is generally a space that is wide and thin.

Also, in addition to the position information obtained by a high-precision GPS, the MMS can use information relating to the travel distance obtained based on the rotation rate of a tire (or angle of a tire), information relating to a direction obtained from a gyroscope, map information accumulated in advance (e.g., information held by a car navigation system), and the like to perform correction of the coordinate values of the coordinates indicated by the position data. For this reason, it can be said that the relative positions hardly vary with respect to each other at the positions respectively indicated by all of the position data obtained by the vehicle 1 performing one instance of travel (i.e., e.g., the point group data obtained in the outbound trip travel or the point group data obtained in the return trip travel).

That is, it can be said that a case such as only the measurement range of a portion of the point group data expanding or contracting hardly occurs in the entire measurement range (the above-described space that is wide and thin) obtained based on the point group data obtained by the vehicle 1 performing one instance of traveling. For this reason, for example, only positional misalignment resulting from translation and positional misalignment resulting from rotation occur between the measurement positions of the point group data obtained through the outbound trip travel and the measurement positions of the point group data obtained through the return trip travel.

For this reason, as long as there are at least three feature points that are not on the same straight line, one wide and thin space can be specified by overlaying the point group data obtained through the outbound trip travel (wide and thin space) and the point group data obtained through the return trip travel (wide and thin space) on each other. For this reason, at least three feature points that are not on the same straight line are used.

In FIGS. 29 and 30, as can be understood by viewing the one-dot chain lines connecting the three feature points (the corner of building A, the trunk of a tree in the park, and the corner of building B), in the example shown in FIGS. 29 and 30, it can be understood that the three feature points are not present on the same straight line. Note that the greater the surface area of the polygonal shape surrounded by such one-dot chain lines is, the more the precision of correcting the error of the coordinate values of the coordinates indicated by the point group data is improved.

Point Group Data Overlaying Processing

Hereinafter, point group data overlaying processing will be described in further detail.

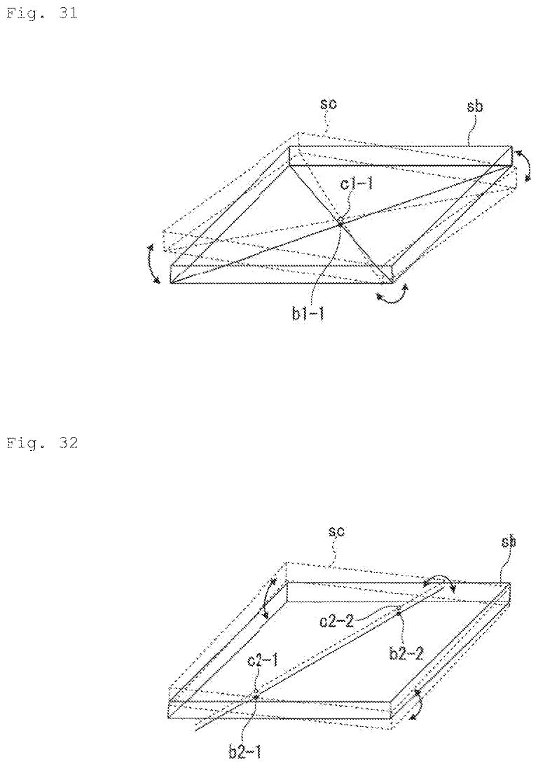

FIGS. 31 to 34 are schematic diagrams for illustrating point group data overlaying processing.

FIG. 31 is a diagram showing point group data overlaying processing performed in the case where only one feature point has been selected. In FIG. 31, the wide and thin space composed of the point group data obtained through the outbound trip travel is a space sb. Also, the wide and thin space composed of point group data obtained through the return trip travel is a space sc. Also, the position data corresponding to the selected feature point out of the point group data obtained through the outbound trip travel is feature point data b1-1. Also, the position data corresponding to the selected feature point out of the point group data obtained through the return trip travel is feature point data c1-1.

As shown in FIG. 31, the MMS overlays the space sb and the space sc on each other by matching the position of the coordinates indicated by the feature point data b1-1 and the position of the coordinates indicated by the feature point data c1-1 with each other. In this case, if, for example, the space sb is used as a reference, the space sc can rotate in either direction centered about the coordinates indicated by the feature point data b1-1 (i.e., the coordinates indicated by the feature point data c1-1 as well). For this reason, the MMS cannot uniquely specify the position (orientation) of the space sc. Accordingly, if only one feature point has been selected, the MMS cannot perform correction of the point group data with high precision.

FIG. 32 is a diagram showing point group data overlaying processing performed in the case where two feature points have been selected. In FIG. 32, the wide and thin space composed of the point group data obtained through the outbound trip travel is a space sb. Also, the wide and thin space composed of point group data obtained through the return trip travel is a space sc. Also, the position data corresponding to the selected feature points out of the point group data obtained through the outbound trip travel is feature point data b2-1 and feature point data b2-2. Also, the position data corresponding to the selected feature points out of the point group data obtained through the return trip travel is feature point data c2-1 and feature point data c2-2.

As shown in FIG. 32, the MMS overlays the space sb and the space sc on each other by matching the position of the coordinates indicated by the feature point data b2-1 and the position of the coordinates indicated by the feature point data c2-1 with each other, and matching the position of the coordinates indicated by the feature point data b2-2 and the position of the coordinates indicated by the feature point data c2-2 with each other. That is, the MMS overlays the space sb and the space sc on each other by matching the straight line connecting the coordinates indicated by the feature point data b2-1 and the coordinates indicated by the feature point data b2-2 and the straight line connecting the coordinates indicated by the feature point data c2-1 and the coordinates indicated by the feature point data c2-2 with each other.

In this case, if, for example, the space sb is used as a reference, the space sc can rotate centered about the straight line connecting the coordinates indicated by the feature point data b2-1 and the coordinates indicated by the feature point data b2-2 (i.e., the straight line connecting the coordinates indicated by the feature point data c2-1 and the coordinates indicated by the feature point data c2-2 as well). For this reason, the MMS cannot uniquely specify the position (orientation) of the space sc. Accordingly, if two feature points have been selected, the MMS cannot perform correction of the point group data with high precision.

FIG. 33 is a diagram showing point group data overlaying processing performed in the case where three feature points have been selected and the positions of the three selected feature points are arranged side by side on the same straight line. In FIG. 33, the wide and thin space composed of the point group data obtained through the outbound trip travel is a space sb. Also, the wide and thin space composed of point group data obtained through the return trip travel is a space sc. Also, the position data corresponding to the selected feature points out of the point group data obtained through the outbound trip travel is feature point data b3-1, feature point data b3-2, and feature point data b3-3. Also, the position data corresponding to the selected feature points out of the point group data obtained through the return trip travel is feature point data c3-1, feature point data c3-2, and feature point data c3-3.

As shown in FIG. 33, the MMS overlays the space sb and the space sc on each other by matching the position of the coordinates indicated by the feature point data b3-1 and the position of the coordinates indicated by the feature point data c3-1 with each other, matching the position of the coordinates indicated by the feature point data b3-2 and the position of the coordinates indicated by the feature point data c3-2 with each other, and matching the position of the coordinates indicated by the feature point data b3-3 and the position of the coordinates indicated by the feature point data c3-3 with each other. That is, the MMS overlays the space sb and the space sc on each other by matching a straight line connecting the coordinates indicated by the feature point data b3-1, the coordinates indicated by the feature point data b3-2, and the coordinates indicated by the feature point data b3-3 and a straight line connecting the coordinates indicated by the feature point data c3-1, the coordinates indicated by the feature point data c3-2, and the coordinates indicated by the feature point data c3-3 with each other.

In this case, similarly to FIG. 32, if, for example, the space sb is used as a reference, the space sc can rotate centered about the straight line connecting the coordinates indicated by the feature point data b3-1, the coordinates indicated by the feature point data b3-2, and the coordinates indicated by the feature point data b3-3 (i.e., the straight line connecting the coordinates indicated by the feature point data c3-1, the coordinates indicated by the feature point data c3-2, and the coordinates indicated by the feature point data c3-3 as well). For this reason, the MMS cannot uniquely specify the position (orientation) of the space sc. That is, even if three feature points are selected, if the positions of the three feature points are on the same straight line, it is not possible to fix the position of the space sc. Accordingly, the MMS cannot perform correction of the point group data with high precision.

On the other hand, in the following conditions, the MMS can correct all of the point group data with high precision by overlaying the space sb and the space sc on each other.

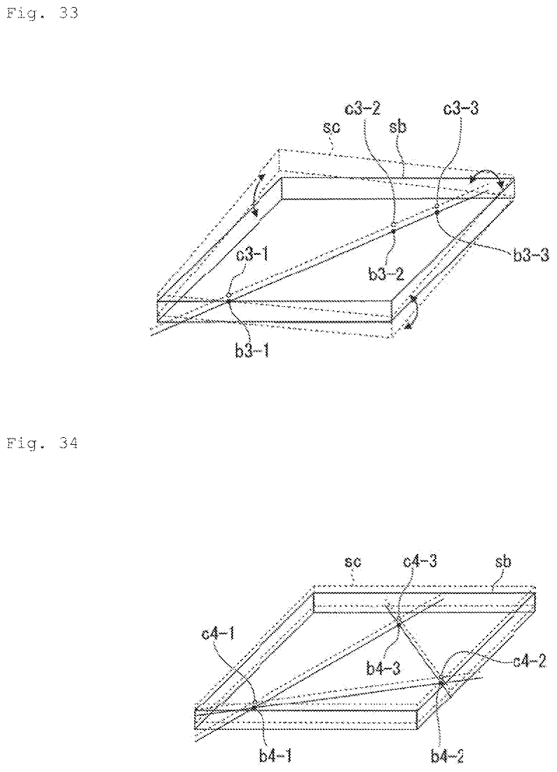

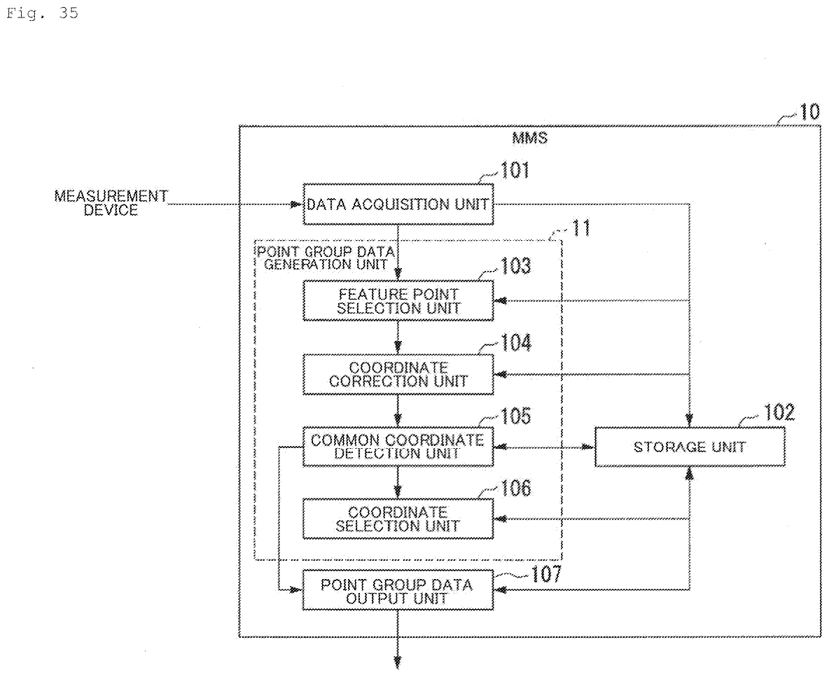

FIG. 34 is a diagram showing point group data overlaying processing performed in the case where three feature points have been selected and the positions of the three selected feature points are not arranged side by side on the same straight line. In FIG. 34, the wide and thin space composed of the point group data obtained through the outbound trip travel is a space sb. Also, the wide and thin space composed of point group data obtained through the return trip travel is a space sc. Also, the position data corresponding to the selected feature points out of the point group data obtained through the outbound trip travel is feature point data b4-1, feature point data b4-2, and feature point data b4-3. Also, the position data corresponding to the selected feature points out of the point group data obtained through the return trip travel is feature point data c4-1, feature point data c4-2, and feature point data c4-3.

As shown in FIG. 34, the MMS overlays the space sb and the space sc on each other by matching the position of the coordinates indicated by the feature point data b4-1 and the position of the coordinates indicated by the feature point data c4-1 with each other, matching the position of the coordinates indicated by the feature point data b4-2 and the position of the coordinates indicated by the feature point data c4-2 with each other, and matching the position of the coordinates indicated by the feature point data b4-3 and the position of the coordinates indicated by the feature point data c4-3 with each other. That is, the MMS overlays the space sb and the space sc on each other by matching a plane formed by connecting the coordinates indicated by the feature point data b3-1, the coordinates indicated by the feature point data b3-2, and the coordinates indicated by the feature point data b3-3 to each other by straight lines, and a plane formed by connecting the coordinates indicated by the feature point data c3-1, the coordinates indicated by the feature point data c3-2, and the coordinates indicated by the feature point data c3-3 to each other by straight lines, with each other.

In this case, matching the plane formed by connecting the coordinates indicated by the feature point data b3-1, the coordinates indicated by the feature point data b3-2, and the coordinates indicated by the feature point data b3-3 to each other by straight lines, and the plane formed by connecting the coordinates indicated by the feature point data c3-1, the coordinates indicated by the feature point data c3-2, and the coordinates indicated by the feature point data c3-3 to each other by straight lines, with each other is, in other words, synonymous with matching the position of the space sb and the position of the space sc with each other. For this reason, if, for example, the space sb is used as a reference, the space sc cannot perform rotation or translation, and the MMS can uniquely specify the position (orientation) of the space sc. That is, if three feature points have been selected and the positions of the three selected feature points are not on the same straight line, the position of the space sc is fixed. Accordingly, the MMS can perform correction of all of the point group data with high precision.

In the description above, point group data overlaying processing performed in respective cases was described with reference to FIGS. 31 to 34 using four methods of selecting the feature points. To summarize below, the MMS cannot correct all of the point group data with high precision in the case of the following conditions 1 to 3. On the other hand, the MMS can correct all of the point group data with high precision in the case of the following condition 4.

Condition 1: Point group data overlaying processing is performed with only one feature point selected (FIG. 31).

Condition 2: Point group data overlaying processing is performed with two feature points selected (FIG. 32).
Condition 3: Point group data overlaying processing is performed with three feature points on one straight line selected (FIG. 33).
Condition 4: Point group data overlaying processing is performed with three feature points that are not on one straight line selected (FIG. 34).

In this manner, in the case of any of conditions 1 to 3, it can be understood that even if the coordinates indicated by the pieces of feature point data are matched with each other in the space sb and the space sc, the space sb composed of the point group data obtained through the outbound trip travel and the space sc composed of the point group data obtained through the return trip travel are not necessarily overlaid on each other. On the other hand, in the case of condition 4, it can be understood that if the coordinates indicated by the pieces of feature point data are matched with each other in the space sb and the space sc, the space sb composed of the point group data obtained through the outbound trip travel and the space sc composed of the point group data obtained through the return trip travel are overlaid on each other.

Accordingly, by matching the position of the space sb and the position of the space sc with each other, for example, the MMS can correct the coordinate values of the coordinates indicated by the position data obtained for the position data acquisition positions Pc in the return trip travel with high precision using, as a reference, the coordinate values of the coordinates indicated by the position data obtained for the position data acquisition positions Pb in the outbound trip travel.

Note that the processing using the point group data generated as described above can be applied not only in measurement of objects that are present in the surrounding area of a road such as that of the present embodiment, but also in various technical fields. For example, the processing can be applied also to pole detection processing (e.g., see PTL 1), communication cable detection processing (e.g., see PTL 2), road sign detection processing (e.g., see PTL 3), and the like.

Note that if the coordinates indicated by the position data obtained on the outbound trip and the coordinates indicated by the position data obtained on the return trip are present near each other, the reason why the MMS according to the present embodiment leaves only one piece of position data and deletes the other piece of position data is as follows.

In general, the MMS often performs processing on a large amount of position data at once. If the coordinated indicated by two pieces of position data are present near each other as described above, the MMS according to the present embodiment deletes one piece of the position data. That is, these two pieces of position data are regarded as being the same information, and are combined into one piece of position data. First, a first reason is so that the data amount of the point group data is reduced and the load of processing in the MMS is reduced by combining these two pieces of position data into one piece of position data.

Also, the point group data obtained through the outbound trip travel and the return trip travel includes both position data obtained overlapping with the same (or nearly the same) coordinates, and position data obtained through either one of the outbound trip travel and the return trip travel. Accordingly, if, for example, an apparatus or the like for performing subsequent processing uses the point group data output from the MMS, an inconvenience occurs in some cases. For example, since information corresponding to the volume is generally not included in the point group data, processing for weighting the density at which the position data is to be acquired for each region to be measured (e.g., in the present embodiment, according to the difference in the sizes of the above-described measurable spaces that are different for each region).

However, if processing for deleting one piece of position data is not performed if coordinates indicated by two pieces of position data are present near each other, such as those described above, the MMS needs to separately perform processing such as performing mutually different weightings on the position data acquired overlapping at the same (or nearly the same) coordinates, and the position data acquired through only one of the outbound trip travel or the return trip travel. A second reason is so that the load on the MMS is reduced in the processing for weighting the density at which such position data is to be acquired.

Note that as described above, for example, the determination indicated in steps S105 and S106 in FIG. 28 (i.e., the determination of whether or not the two coordinate values are similar) is performed according to, for example, whether or not the distance is less than the interval of the resolution of the laser radar. Here, the interval of the resolution corresponds to the size of the measurable space, which was described with reference to FIGS. 7 and 8. That is, for example, if the acquisition positions of the position data in the return trip travel (the position data acquisition positions Pc) are included in the above-described measurable space in which the acquisition positions of the position data in the outbound travel (position data acquisition positions Pb) are included, it is determined that the two coordinate values are similar.

Functional Configuration of MMS

Hereinafter, a configuration of an MMS will be described.

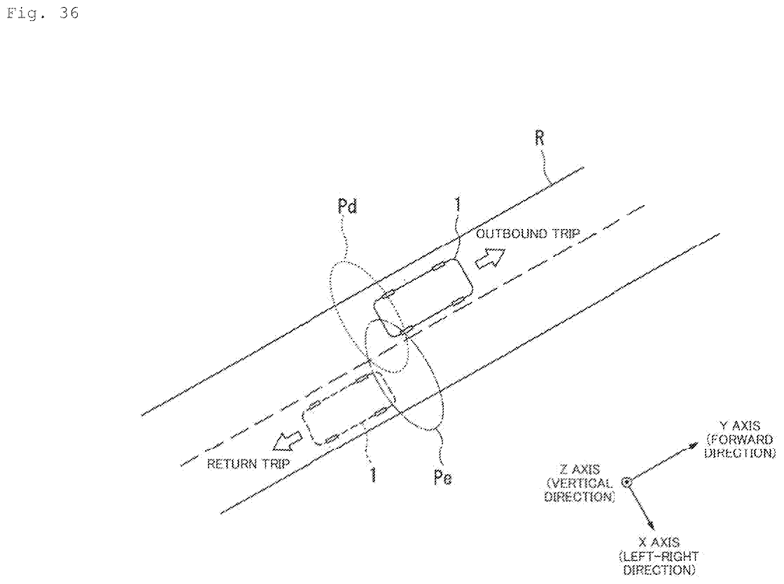

FIG. 35 is a block diagram showing a functional configuration of an MMS 10 according to a first embodiment of the present invention. The MMS 10 is, for example, a measurement apparatus constituted by an information processing apparatus such as a general-purpose computer. The MMS 10 is mounted in, for example, a moving body such as the above-described vehicle 1.

Note that the moving body moves round-trip on the same moving route. The moving body is equipped with, for example, a measurement device (not shown) such as a laser radar. The measurement device measures the position of an object in the periphery of the movement route of the moving body and generates position data. The position data generated here includes the position data obtained on the outbound trip and the position data obtained on the return trip. Note that the measurement device performs position measurement for the position data acquisition positions (e.g., the above-described position data acquisition positions Pb and position data acquisition positions Pc) that are present on the measurement plane that has a depression angle (or an elevation angle) in the forward direction of the moving body with respect to the plane (lateral cross-section) orthogonal to the forward direction of the moving body. The measurement device outputs the position data obtained based on the measurement results to the MMS 10.

The MMS 10 acquires the position data output from the measurement device. The MMS 10 processes the position data so as to align the position data obtained on the outbound trip and the position data obtained on the return trip with each other. Accordingly, the point group data, which is a set of the processed position data output from the MMS 10, is data that can be used by a general apparatus for performing various types of subsequent processing using only the point group data composed of the position data obtained on the outbound trip.

As shown in FIG. 35, the MMS 10 is constituted by including a point group data generation unit 11, a data acquisition unit 101, a storage unit 102, and a point group data output unit 107. Also, the point group data generation unit 11 is constituted by including a feature point selection unit 103, a coordinate correction unit 104, a common coordinate detection unit 105, and a coordinate selection unit 106.

From an external apparatus or the like that includes, for example, a measurement device such as a laser radar, the data acquisition unit 101 acquires the position data obtained by the measurement device during outbound trip movement of the moving body and the position data obtained by the measurement device during return trip movement of the moving body. The data acquisition unit 101 stores the acquired position data in the storage unit 102.

The storage unit 102 stores the point group data, which is a set of position data. The storage unit 102 is realized by, for example, a storage medium such as a flash memory, an HDD (Hard Disk Drive), an SDD (Solid State Drive), a RAM (Random Access Memory; a readable/writable memory), an EEPROM (Electrically Erasable Programmable Read Only Memory), or a register, or a combination of these storage mediums.

The point group data generation unit 11 generates the point group data based on the position data acquired by the data acquisition unit 101.

When the position data is stored in the storage unit 102 by the data acquisition unit 101, the feature point selection unit 103 selects three or more feature points from the position data selected in the storage unit 102. For example, the feature point selection unit 103 selects, as the feature points, position data corresponding to the vicinity of the departure point of the outbound trip, the vicinity of a point on the movement path, and the vicinity of the departure point of return trip in the periphery of the movement path of the moving body. Note that at this time, the feature point selection unit 103 selects the feature points such that the positions of all of the feature points are not positions on the same straight line. The feature point selection unit 103 outputs information indicting the selected feature points to the coordinate correction unit 104.

The coordinate correction unit 104 acquires the information indicating the selected feature points that was output from the feature point selection unit 103. The coordinate correction unit 104 extracts the position data corresponding to the feature points in the position data obtained on the outbound trip and the position data corresponding to the feature points in the position data obtained on the return trip from the position data stored in the storage unit 102 based on the acquired information. For each feature point, the coordinate correction unit 104 performs coordinate alignment processing for matching the outbound trip coordinates indicated by the position data obtained on the outbound trip and the return trip coordinates indicated by the position data obtained on the return trip with each other. The coordinate correction unit 104 corrects the coordinate values of the coordinates indicated by the position data other than the feature points based on the coordinate values of the coordinates indicated by the position data of the feature points subjected to the coordinate alignment processing. The coordinate correction unit 104 updates the position data stored in the storage unit 102 to the corrected position data.

When the correction processing of the position data performed by the coordinate correction unit 104 is complete, the common coordinate detection unit 105 references the position data stored in the storage unit 102 and executes detection of two pieces of position data for which the coordinate values are similar to each other in the point group data of the outbound trip and the point group data of the return trip. If two pieces of position data whose coordinate values are similar to each other are detected, the common coordinate detection unit 105 outputs the information indicating the set of these two pieces of position data to the coordinate selection unit 106.

The coordinate selection unit 106 acquires information indicating the set of two pieces of position data output from the common coordinate detection unit 105. Based on the acquired information, the coordinate selection unit 106 calculates the size of the space that can be measured by the measurement device used in the measurement based on the respective coordinates indicated by the two pieces of position data (i.e., the size of the smallest measurement target that is actually subjected to the measurement by the measurement device) and compares both of the measurement results. Based on the result of the comparison processing, the coordinate selection unit 106 deletes the position data for which the size of the measurable space is larger out of these two pieces of position data stored in the storage unit 102.

The common coordinate detection unit 105 executes the above-described processing of the coordinate selection unit 106 until the two pieces of position data whose coordinate values are similar to each other are no longer detected in the point group data stored in the storage unit 102. If two pieces of position data whose coordinate values are similar to each other are not detected in the point group data stored in the storage unit 102, the common coordinate detection unit 105 outputs instruction information indicating a point group data output instruction to the point group data output unit 107.

Upon obtaining the instruction information from the common coordinate detection unit 105, the point group data output unit 107 outputs the point group data stored in the storage unit 102 to an external apparatus.

As described above, the MMS 10 according to the first embodiment of the present invention acquires the position data for the position data acquisition positions (e.g., the above-described position data acquisition positions Pb and the position data acquisition positions Pc) that are present on the measurement plane having a depression angle (or elevation angle) in the forward direction of the vehicle 1 with respect to the plane (lateral cross-section) that is orthogonal to the forward direction of the vehicle 1. Also, the MMS 10 acquires the position data obtained on the outbound trip and the position data on the return trip using the vehicle 1 that travels round-trip on a travel path. The MMS 10 enables measurement also for the measurement-undetected regions that conventionally could not be measured by acquiring and analyzing the position data obtained as described above. This makes it possible for the MMS 10 according to the present embodiment to reduce measurement-undetected regions.

Also, as described above, the vehicle 1 travels round-trip on the same road, and the MMS 10 according to the present embodiment executes the processing shown in the flowchart of FIG. 28 on the position data obtained on the outbound trip and the return trip. According to this processing, if the coordinates indicated by the position data obtained on the outbound trip and the coordinates indicated by the position data obtained on the return trip are near each other, only one piece of position data is selected and the other is deleted. Accordingly, the point group data, which is a set of position data processed by the MMS 10 according to the present embodiment, can be used also by a conventional apparatus for performing various types of subsequent processing based on the point group data composed of only position data obtained in the outbound travel.

### Second Embodiment

Hereinafter, a second embodiment of the present invention will be described with reference to the drawings.

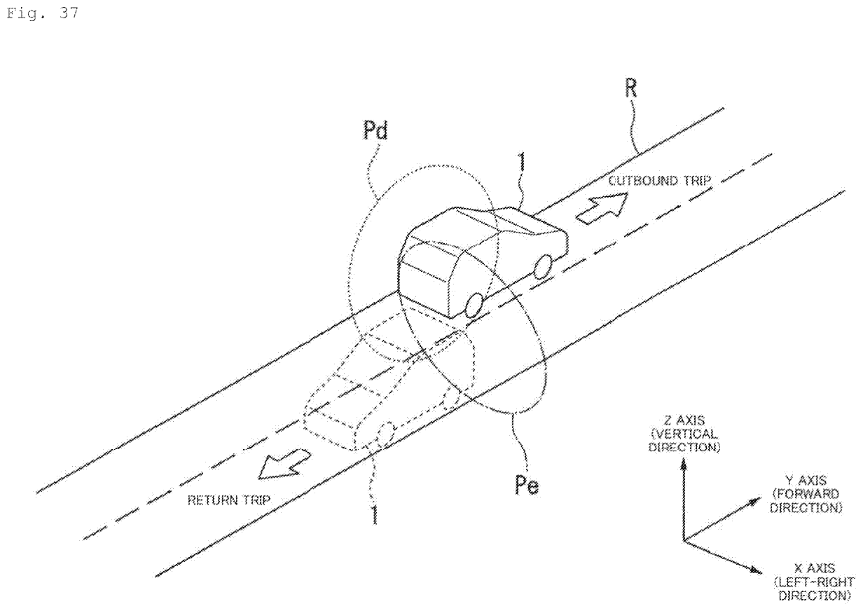

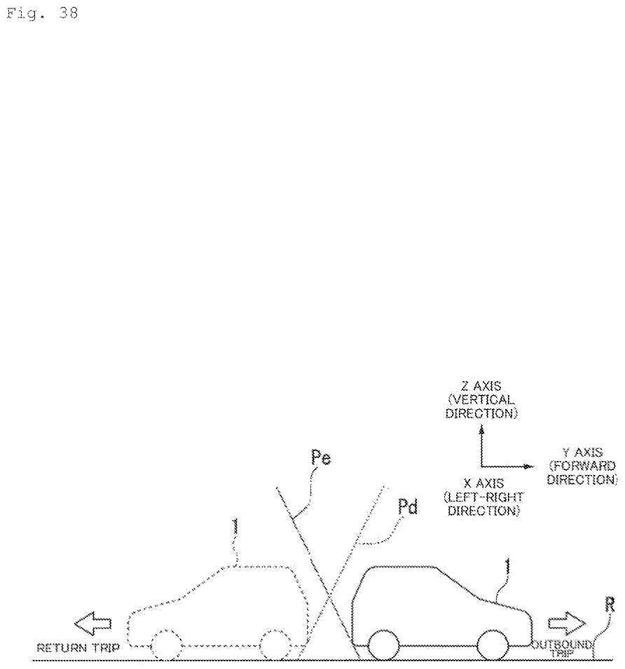

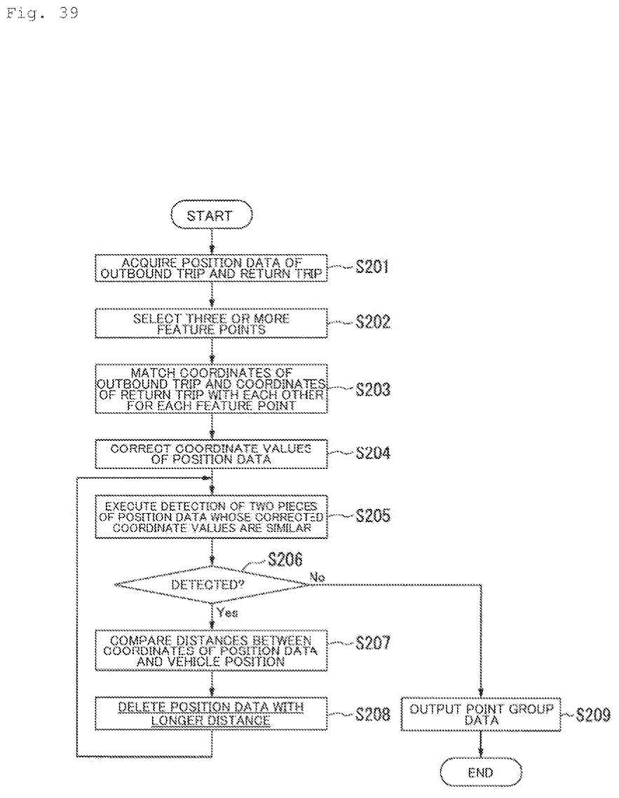

FIGS. 36 to 38 are schematic diagrams showing acquisition positions of position data acquired by an MMS according to a second embodiment of the present invention. In the present embodiment, a vehicle 1 travels round-trip on a road R. However, as can be understood from FIGS. 36 and 37, in the present embodiment, unlike in the above-described first embodiment, the vehicle 1 travels in different travel lanes on the outbound trip and the return trip. That is, on the outbound trip and the return trip as well, the vehicle 1 travels in the lane on the left side on the road R with respect to the forward direction. FIGS. 36 to 38 respectively show a plan view, a bird's-eye view, and a vertical cross-sectional view of a space in which the vehicle 1 in which the MMS is mounted is present, at one point in time on an outbound trip and at one point in time on a return trip. Hereinafter, description will be given with a focus on points in which the configuration differs from the first embodiment.

Due to the travel lanes in which the vehicle 1 travels being different on the outbound trip and the return trip, as shown in FIGS. 36 and 37, the position data acquisition positions on the outbound trip (position data acquisition positions Pd) and the position data acquisition positions on the return trip (position data acquisition positions Pe) are positions that are shifted with respect to each other in the left-right direction (x axis direction) of the vehicle 1. That is, even though the measurement target is the same, the distance from the position of the laser radar to the position of the measurement target is different on the outbound trip and the return trip.

However, the measurement plane including the position data acquisition position Pd and the measurement plane including the position data acquisition position Pe are respectively present on the same planes as the measurement plane including the position data acquisition position Pb and the measurement plane including the position data acquisition position Pc in the above-described first embodiment. This makes it possible for the MMS according to the second embodiment to measure the measurement-undetected regions that conventionally could not be measured as well by acquiring and analyzing the position data obtained on the outbound trip and the position data obtained on the return trip. This makes it possible for the MMS according to the present embodiment to reduce the measurement-undetected regions.

Also, similarly to the above-described first embodiment, in the present embodiment as well, the greater the difference between the height of the position of the leaf Lf and the height of the position of the laser radar is, the more the component in the forward direction or the opposite direction of the vehicle 1 (the frontward direction or the rearward direction in the y axis direction), which is included in the emission direction of the laser light, is included, and therefore the likelihood that the laser light will hit the leaf Lf becomes greater.

For example, if the laser radar is mounted on the roof of the vehicle 1, the position data acquisition positions Pd or Pe acquired by the MMS are located on the measurement plane having a depression angle (or an elevation angle) in the forward direction (y axis direction) instead of on the lateral cross-section (x-z plane), which is a plane that is orthogonal to the forward direction (y axis direction) of the vehicle 1. Accordingly, the emission direction of the laser light emitted to the measurement target located at a position higher than the height of the roof of the vehicle position (i.e., the installation height of the laser radar) includes a component in the opposite direction (the plus direction in the y axis direction) of the forward direction of the vehicle 1. On the other hand, the emission direction of the laser light emitted to the measurement target located at a position lower than the height of the roof of the vehicle position includes a component in the forward direction of the vehicle 1 (the minus direction in the y axis direction).

Specifically, if the laser light is emitted to a position at the same height as the position of the laser radar, the laser light is emitted in a direction (X axis direction) that is orthogonal to the forward direction of the vehicle 1. In contrast to this, if the laser light is emitted to a position that is higher than the position of the laser radar, the laser light is emitted in a direction that is obliquely upward and rearward of the vehicle 1 (if the measurement plane has a depression angle). Also, if the laser light is emitted to a position that is lower than the position of the laser radar, the laser light is emitted in a direction that is obliquely downward and frontward of the vehicle 1 (if the measurement plane has a depression angle). Accordingly, even if the leaf Lf is growing with its side surface facing the vehicle 1, if the heights of the position of the leaf Lf and the position of the laser radar are different from each other, the laser light is emitted in the direction including a component in the frontward direction or the rearward direction of the vehicle 1, and therefore the likelihood that the laser light will hit increases.

Operation of MMS

Hereinafter, an example of operations of the MMS (measurement apparatus) will be described.

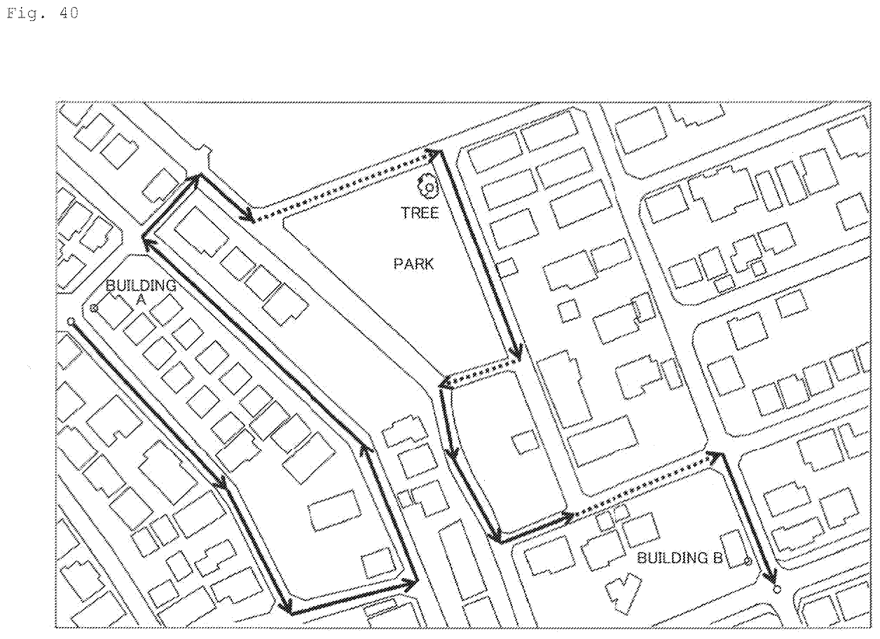

FIG. 39 is a flowchart showing operations of the MMS according to the second embodiment of the present invention. Note that the processing from the step S201 to step S206 and the processing of step S209 shown in FIG. 39 are the same as the processing from the step S101 to step S106 and the processing of step S109 in the flowchart indicating the operations of the MMS according to the first embodiment shown in FIG. 28, and therefore description thereof is omitted.

If two pieces of position data whose corrected coordinate values are similar to each other are detected in the position data of the outbound trip and the position data of the return trip (step S206: Yes), the MMS calculates the distance between the coordinates indicated by one piece of position data (e.g., the position data of the outbound trip) and the position of the laser radar at the point in time when the measurement for that position data was performed and the distance between the coordinates indicated by the other piece of position data (e.g., the position data of the return trip) and the position of the laser radar at the point in time when the measurement for that position data was performed. The MMS compares both distances, which are the calculation results (step S207).

The MMS deletes the position data for which the calculated distance is longer out of the two pieces of position data based on the result of the above-described comparison processing (step S208).

Functional Configuration of MMS

Hereinafter, the configuration of the MMS will be described.

The block diagram showing the functional configuration of the MMS according to the second embodiment is the same as the block diagram showing the functional configuration of the MMS 10 according to the first embodiment shown in FIG. 35. The MMS according to the second embodiment and the MMS 10 according to the above-described first embodiment differ only in the functional configuration of the coordinate selection unit 106.

The coordinate selection unit 106 according to the present embodiment acquires information indicating a set of two pieces of position data output from the common coordinate detection unit 105. The coordinate selection unit 106 calculates the distance between the coordinates indicated by one piece of position data (e.g., the position data of the outbound trip) and the position of a measurement device (not shown) at the point in time when the measurement for that position data was performed, and the distance between the coordinates indicated by the other piece of position data (e.g., the position data of the return trip) and the position of a measurement device (not shown) at the point in time when the measurement for that position data was performed. Then, the coordinate selection unit 106 compares both distances, which are the calculation results.

Based on the result of the comparison processing, the coordinate selection unit 106 deletes the position data for which the calculated distance is longer out of these two pieces of position data stored in the storage unit 102.

As described above, the MMS according to the first embodiment performs processing for comparing the sizes of the spaces that can be measured by the MMS in the two pieces of position data whose coordinate values are similar to each other, and deleting the position data with the greater size. In contrast to this, the MMS according to the second embodiment deletes the position data with a longer distance between the coordinates indicated by the position data and the position of the laser radar at the point in time when the measurement for that position data was performed, with respect to the two pieces of position data whose coordinate values are similar to each other. That is, in the second embodiment, the position data that was acquired when the vehicle 1 was traveling in the travel lane closer to the coordinates of these two pieces of position data (the travel lane on the side on which the coordinates are present in a view from the road R) is left.

As described above, the MMS according to the second embodiment of the present invention can perform selection processing of the two pieces of position data whose coordinate values are similar to each other in order to calculate the distance between the coordinates indicated by the position data and the laser radar without calculating the size of the space that can be measured by the MMS. This makes it possible for the MMS according to the present embodiment to more easily and more quickly perform processing of the position data.

Also, as described above, the MMS according to the second embodiment of the present invention acquires the position data for the position data acquisition positions (e.g., the above-described position data acquisition positions Pd and position data acquisition positions Pe) that are present on the measurement plane having a depression angle (or an elevation angle) in the forward direction of the vehicle 1 with respect to the plane that is orthogonal to the forward direction of the vehicle 1. Also, the MMS acquires the position data acquired on the outbound trip and the position data of the return trip using the vehicle 1 that travels round-trip on the travel route. The MMS can also measure the measurement-undetected regions that conventionally could not be measured, by acquiring and analyzing the above-described position data. This makes it possible for the MMS according to the present embodiment to reduce the measurement-undetected regions.

Also, as described above, the vehicle 1 travels round-trip on the same road, and the MMS according to the present embodiment executes the processing shown in the flowchart of FIG. 39 on the position data obtained on the outbound trip and the return trip. According to this processing, if the coordinates indicated by the position data obtained on the outbound trip and the coordinates indicated by the position data obtained on the return trip are near each other, only one piece of position data is selected and the other is deleted. Accordingly, the point group data, which is a set of position data processed by the MMS according to the present embodiment, can be used also by a conventional apparatus for performing various types of subsequent processing based on the point group data composed of only position data obtained in the outbound travel.

### Third Embodiment

Hereinafter, a third embodiment of the present invention will be described with reference to the drawings.

In the above-described second embodiment, the vehicle 1 was configured to travel in different travel lanes on the same road R on the outbound trip and the return trip. On the other hand, in the present embodiment, the vehicle 1 travels on different travel routes (different roads) on the outbound trip and the return trip.

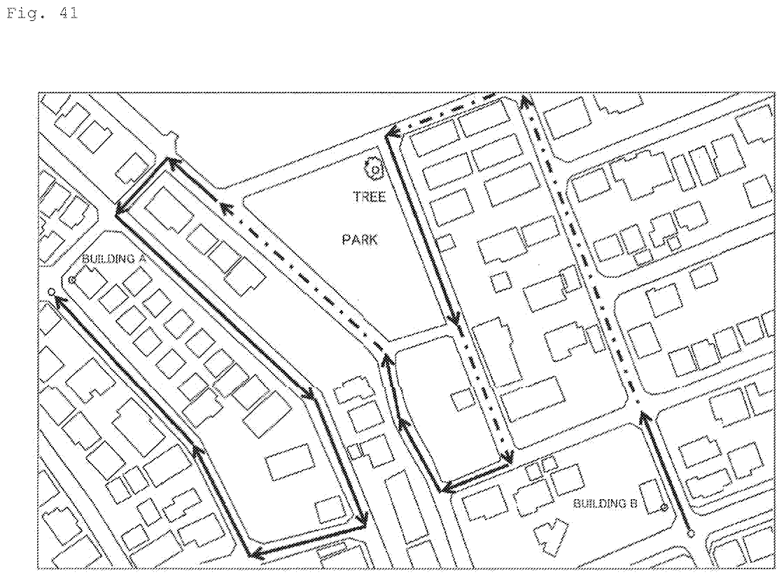

FIGS. 40 and 41 are schematic diagrams for illustrating coordinate alignment processing performed by the MMS according to the third embodiment of the present invention.

FIG. 40 shows the travel route of the outbound trip of the vehicle 1 and the three selected feature points on a map. On the other hand, FIG. 41 shows the travel route of the return trip of the vehicle 1 and the three selected feature points on a map.

On the travel route of the outbound trip shown in FIG. 40, the first feature point is a portion of a corner of a building A that is present near the departure point of the outbound trip of the vehicle 1. Also, the second feature point is a trunk of a tree that is present in a park on the travel route. Also, the third feature point is a portion of a corner of a building B that is present near the arrival point of the outbound trip of the vehicle 1 (i.e., the departure point of the return trip).

On the other hand, on the travel route of the return trip shown in FIG. 41, the first feature point is a portion of a corner of the building B that is present near the departure point of the return trip of the vehicle 1. Also, the second feature point is a trunk of a tree that is present in a park on the travel route. Also, the third feature point is a portion of a corner of a building A that is present near the arrival point of the return trip of the vehicle 1 (i.e., the departure point of the outbound trip).

As can be understood by comparing FIGS. 40 and 41, the travel route of the outbound trip and the travel route of the return trip of the vehicle 1 are different from each other. In FIG. 40, the travel sections denoted by the dotted-line arrows are sections in which the vehicle 1 travels only on the outbound trip. On the other hand, in FIG. 41, the travel sections denoted by the one-dot chain line arrows are sections in which the vehicle 1 travels only on the return trip.

Similarly to the above-described first embodiment, the MMS overlays the point group data obtained on the outbound trip and the point group data obtained on the return trip on each other by matching the coordinates of the position data obtained on the outbound trip and the coordinates of the position data obtained on the return trip with each other, the coordinates of the position data corresponding to three feature points (the corner of building A, the trunk of the tree in the park, and the corner of building B in FIGS. 40 and 41). Then, similarly to the first embodiment, the MMS performs correction on all of the point group data.

Then, the MMS according to the present embodiment executes coordinate alignment processing that is the same as the coordinate alignment processing performed by the MMS according to the above-described first embodiment or the second embodiment only for the travel section in which the vehicle 1 travels round-trip (i.e., only for the travel section indicated by the solid-line arrow in FIGS. 40 and 41).

Also, the MMS according to the present embodiment outputs the travel sections in which the vehicle 1 travels only on one of the outbound trip and the return trip (i.e., the travel sections indicated by the dotted-line arrows in FIG. 40 and the travel section indicated by the one-dot chain line arrows in FIG. 41) as-is without performing the above-described coordinate alignment processing. This is because it is thought that, regarding the travel sections in which the vehicle 1 travels on only one of the outbound trip and the return trip, essentially, there are not two pieces of position data whose coordinate values are similar to each other in the point group data of the outbound trip and the point group data of the return trip.

In this manner, in the third embodiment of the present invention, the coordinate alignment processing is not performed for the travel sections in which the vehicle 1 travels on only one of the outbound trip and the return trip. Accordingly, with the present embodiment, similarly to the first embodiment and the second embodiment, the measurement-undetected regions are reduced, and the load in the coordinate alignment processing of the MMS is reduced.

As described above, the MMS in the above-described first to third embodiments performs pre-processing on the point group data to be used in the apparatus for performing subsequent processing, and provides the point group data for which the measurement-undetected regions have been further reduced to the apparatus for performing subsequent processing. Accordingly, the processing precision of the processing to be performed in a downstream apparatus that is to use the point group data (e.g., processing for extracting various types of objects in the periphery of the road from the point group data, processing for inspecting the extracted objects, etc.) further improves.

Note that the MMS in the above-described first to third embodiments need not be an apparatus that is mounted in a moving body. For example, the MMS may also have a configuration of being installed in a room and acquiring position data measured by a measuring device installed in a moving body through wireless communication, or acquiring the position data via a storage medium that is easily attached and removed.

Note that the MMS may also be mounted in the moving body and the laser radar may be built into the MMS.

Note that the MMS in the above-described first to third embodiments can also be realized by a computer and a program. In this case, the program may also be configured to be recorded in a recording medium, and may be configured to a provided via a network.

The MMS of the above-described embodiments may also be realized by a computer. In this case, the MMS may be realized by recording a program for realizing the function in a computer-readable recording medium, loading the program recorded in the recording medium in a computer system, and executing the program. Note that it is assumed that the “computer system” in this context includes an OS and hardware such as a peripheral device. Also, a “computer-readable recording medium” refers to a storage apparatus such as a flexible disk, a magneto-optical disk, a ROM, a portable medium such as a CD-ROM, or a hard disk built into a computer system. Furthermore, a “computer-readable recording medium” may also include a recording medium that dynamically holds a program for a short period of time, as with a communication line used when transmitting a program via a network such as the Internet or a communication line such as a telephone line, and a recording medium that holds a program for a certain amount of time, as with a volatile memory in a computer system that is to be a server or a client in this case. Also, the above-described program may be for realizing some of the above-described functions, may further be able to realize the above-described function in combination with a program that has already been recorded in a computer system, and may be realized using a programmable logic device such as an FPGA (Field Programmable Gate Array).

### REFERENCE SIGNS LIST

1 Vehicle
10 MMS
11 Point group data generation unit
101 Data acquisition unit
102 Storage unit
103 Feature point selection unit
104 Coordinate correction unit
105 Common coordinate detection unit
106 Coordinate selection unit
107 Point group data output unit

1. A measurement method comprising:
a data acquisition step of acquiring position data output from a measurement device configured to move round-trip on a movement route and measure a position of an object in a periphery of the movement route, the measurement device using a plane having a depression angle or an elevation angle with respect to a plane that is orthogonal to a forward direction as a measurement target; and
a point group data generation step of generating point group data for an object in the periphery of the movement path using the position data obtained during movement of the measurement device on an outbound trip and the position data obtained during movement of the measurement device on a return trip.

2. The measurement method according to claim 1,
wherein in the point group data generation step, the point group data is generated using the position data obtained by aligning a position indicated by feature point data obtained during movement of the measurement device on the outbound trip and a position indicated by the feature point data obtained during movement of the measurement device on the return trip, the feature point data indicating position data obtained with respect to a feature point, which is a specific position in the periphery of the movement route.

3. The measurement method according to claim 2, wherein in the point group data generation step, feature point data that indicates at least three said feature points that are not on one straight line is aligned for each said feature point.

4. The measurement method according to claim 1, wherein in the point group data generation step, the point group data is generated by extracting a set of the position data in which the position indicated by the position data obtained during movement of the measurement device on the outbound trip and the position indicated by the position data obtained during movement of the movement device on the return trip are located near each other, and deleting one piece of the position data included in the set of position data in accordance with a predetermined condition.

5. The measurement method according to claim 4, wherein in the point group data generation step, the point group data is generated by deleting the position data for which the size of the smallest space that can be measured by the measurement device at the position indicated by the position data is larger.

6. The measurement method according to claim 4,
wherein in the data acquisition step, the position data output from the measurement device configured to move in mutually-different lanes on the same movement route during movement on the outbound trip and movement on the return trip is acquired, and
in the point group data generation step, the point group data is generated by deleting the position data for which the distance between the position indicated by the position data and the position of the measurement device at the point in time when the position was measured is longer.

7. A measurement apparatus comprising:
a data acquisition unit configured to acquire position data output from a measurement device configured to move round-trip on a movement route and measure a position of an object in a periphery of the movement route, the measurement device using a plane having a depression angle or an elevation angle with respect to a plane that is orthogonal to a forward direction as a measurement target; and
a point group data generation unit configured to generate point group data for an object in the periphery of the movement path using the position data obtained during movement of the measurement device on an outbound trip and the position data obtained during movement of the measurement device on a return trip.

8. A non-transitory computer-readable medium having computer-executable instructions that, upon execution of the instructions by a processor of a computer, cause the computer to execute the measurement method according to claim 1.
